SEQUENCE LISTING (4 sequences)

SEQ ID NO: 1 - RNA, 100 nt, synthetic construct
  misc_feature 1..100: note = sgRNA-sgr1
  modified_base 1..4: mod_base = OTHER; note = phosphorothioate linkage
  modified_base 3: mod_base = cm
  modified_base 1: mod_base = gm
  modified_base 97..100: mod_base = OTHER; note = phosphonoacetate linkage
  source 1..100: mol_type = other RNA
  modified_base 29: mod_base = gm
  modified_base 2: mod_base = OTHER; note = 2-O-methyl adenosine
  modified_base 30: mod_base = cm
  modified_base 31: mod_base = tm
  modified_base 32: mod_base = OTHER; note = 2-O-Methyladenosine
  modified_base 33: mod_base = gm
  modified_base 34..36: mod_base = OTHER; note = 2-O-Methyladenosine
  modified_base 37: mod_base = tm
  modified_base 38: mod_base = OTHER; note = 2-o-methyladenosine
  modified_base 39: mod_base = gm
  modified_base 40: mod_base = cm
  modified_base 69: mod_base = OTHER; note = 2-o-methyladenosine
  modified_base 70: mod_base = cm
  modified_base 71..72: mod_base = tm
  modified_base 73: mod_base = gm
  modified_base 74..78: mod_base = OTHER; note = 2-o-methyladenosine
  modified_base 79: mod_base = gm
  modified_base 80: mod_base = tm
  modified_base 81..82: mod_base = gm
  modified_base 83: mod_base = cm
  modified_base 84: mod_base = OTHER; note = 2-o-methyladenosine
  modified_base 85..86: mod_base = cm
  modified_base 87: mod_base = gm
  modified_base 88: mod_base = OTHER; note = 2-o-methyladenosine
  modified_base 89: mod_base = gm
  modified_base 90: mod_base = tm
  modified_base 91: mod_base = cm
  modified_base 92..93: mod_base = gm
  modified_base 94: mod_base = tm
  modified_base 95: mod_base = gm
  modified_base 96: mod_base = cm
  modified_base 97..100: mod_base = tm
gaccaactttggccgctgtggttttagagctagaaatagcaagttaaaataaggctagtccgttatcaacttgaaaaagtggcaccgagtcggtgctttt

SEQ ID NO: 2 - RNA, 20 nt, synthetic construct
  misc_feature 1..20: note = Mouse TTR sgRNA
  source 1..20: mol_type = other RNA
ttacagccacgtctacagca

SEQ ID NO: 3 - protein, 9 aa, synthetic construct
  REGION 1..9: note = LAGLIDADG (family of homing endonucleases)
LAGLIDADG

SEQ ID NO: 4 - DNA, 22 bp, synthetic construct
  misc_feature 1..22: note = 22 base pair recognition sequence (for I-CreI family of homing endonucleases)
  source 1..22: mol_type = other DNA
caaaacgtcgtgagacagtttg

(19) United States
(12) Patent Application Publication
(10) Pub. No.: US 2025/0367128 A1
(43) Pub. Date: Dec. 4, 2025

(54) BIODEGRADABLE LIPIDOIDS AND COMPOSITIONS AND METHODS OF USE THEREOF FOR LIVER TARGETED DELIVERY

(71) Applicant: The Trustees of the University of Pennsylvania, Philadelphia, PA (US)

(72) Inventors: Michael Mitchell, Philadelphia, PA (US); Lulu Xue, Philadelphia, PA (US); Lili Wang, Phoenixville, PA (US); James M. Wilson, Philadelphia, PA (US); Claude Warzecha, Wallingford, PA (US)

(21) Appl. No.: 18/853,162

(22) PCT Filed: Apr. 13, 2023
(86) PCT No.: PCT/US2023/065720

§ 371 Date: Oct. 1, 2024

Related U.S. Application Data
(60) Provisional application No. 63/330,972, filed on Apr. 14, 2022.
(60) Provisional application No. 63/364,859, filed on May 17, 2022.

(51) Int. Cl. A61K 9/51; A61K 31/7105; A61K 38/46; A61K 48/00

(52) U.S. Cl. CPC A61K 9/5123; A61K 31/7105; A61K 38/465; A61K 48/0058; C12N 2310/20

(57) ABSTRACT

Described are biodegradable lipid nanoparticles (LNPs) comprising biodegradable lipidoids and compositions thereof. In various embodiments, the LNP selectively targets a liver cell. In other aspects, the present invention relates to methods for in vivo delivery of therapeutic nucleic acids to the liver to prevent or treat diseases or disorders using the LNP compositions of the invention.

### CROSS-REFERENCE TO RELATED APPLICATIONS

This application is a National Stage Entry under 35 U.S.C. 371 of International Patent Application No. PCT/US2023/065720, filed Apr. 13, 2023, which claims the benefit under 35 USC 119 (e) of U.S. Provisional Patent Application No. 63/364,859, filed May 17, 2022, and U.S. Provisional Patent Application No. 63/330,972, filed Apr. 14, 2022 which are incorporated herein by reference.

### STATEMENT REGARDING FEDERALLY SPONSORED RESEARCH OR DEVELOPMENT

This invention was made with government support under DP2 TR002776 awarded by National Institutes of Health. The government has certain rights in the invention.

### REFERENCE TO AN ELECTRONIC SEQUENCE LISTING

The contents of the electronic sequence listing (22-10009.PCT-Seq-Listing_UPDATED.xml; Size: 20.6 KB (21,151 bytes); and Date of Creation: Jun. 2, 2025) is herein incorporated by reference in its entirety.

### BACKGROUND OF THE INVENTION

With the development of RNA therapeutics, gene therapy, gene editing technologies, etc., it is necessary to address the challenge of delivering them to cells in a precise and efficient way. Currently, there are three FDA approved/EUA products that utilize lipid nanoparticles (LNPs)-Onpattro (siRNA) and the Pfizer and Moderna mRNA COVID-19 vaccines.

However, the development of mRNA-LNPs system is challenging. For example, one major challenge in the development of mRNA-LNPs systems is the identification of safety and efficacy, which support a sufficiently broad therapeutic index for chronic indications. Unfortunately, improvements in LNPs delivery potency do not always result in a desired therapeutic index since the restriction and reduction in tolerated dose levels. Although non-hydrolysable lipid-like materials have been proved to exhibit a satisfied delivery efficacy, the delivery toxicity still remains. Based on this, some studies developed degradable LNP systems for in vivo RNA therapeutics delivery, but the delivery potency compared with benchmark LNPs, such as C12-200, was not high enough for low dosing and long-term treatment. Therefore, it is still urgent to develop novel LNP delivery systems with both high delivery efficacy and low toxicity.

Thus, there is a need in the art for LNP delivery systems with high delivery efficacy and low toxicity to deliver RNA therapeutics, gene therapy, gene editing technologies, etc., in a precise and efficient way to a cell of interest. The present disclosure satisfies this unmet need.

### SUMMARY OF THE INVENTION

In a first aspect, a composition comprising biodegradable lipid nanoparticles (LNP) useful for delivering a nucleic acid to a liver cell is provided. The LNP is formed from:
(a) at least one ionizable lipid compound having the structure of Formula (IA), Formula (IB), or combinations thereof:

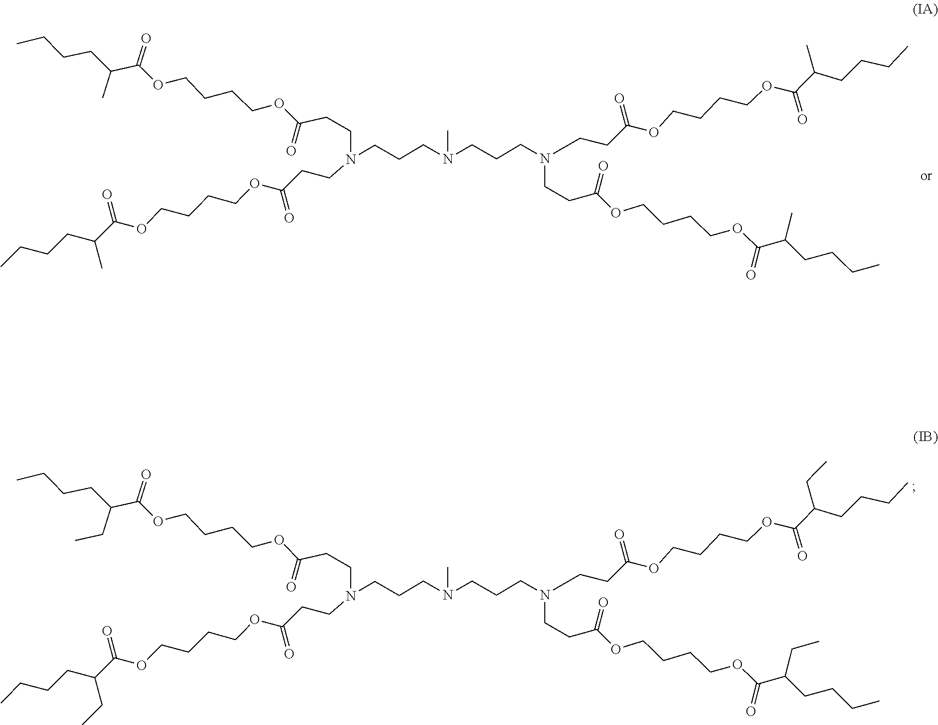

or a racemate, an enantiomer, a diastereomer, a pharmaceutically acceptable salt, or a derivative thereof. The total concentration of ionizable lipid(s) (a) in the LNP is present in a concentration range of about 1 mol % to about 99 mol %, based on the total amount of lipids in the LNP. The LNP further includes (b) at least one neutral phospholipid, wherein the neutral phospholipid is present in a concentration range of about 10 mol % to about 45 mol % based on the total amount of lipids in the LNP; (c) at least one cholesterol lipid, wherein the total cholesterol lipid is in a concentration range of about 5 mol % to about 55 mol % based on the total amount of lipids in the LNP; and (d) at least one polyethylene glycol (PEG) lipid, wherein the total PEG-lipid is in a concentration range of about 0.5 mol % to about 12.5 mol % based on the total amount of lipids in the LNP. In addition, at least one nucleic acid is comprised or encapsulated in the LNP.

In certain embodiments, the nucleic acid comprises a coding sequence for an editing enzyme operably linked to sequences which direct expression thereof in a liver cell. In certain embodiments, the nucleic acid is an mRNA encoding a Cas9. In certain embodiments, the LNP further comprises an sgRNA.

In certain embodiments, the composition further comprises a second nucleic acid that encodes a therapeutic transgene. In certain embodiments, the therapeutic transgene is associated with a liver enzyme disorder, a lysosomal storage disorder, a glycogen storage disease or deficiency, a urea cycle disorder, or a lipid disorder.

In a further aspect, a method of delivering a gene product to a subject in need thereof is provided. The method includes administering a therapeutically effectively amount of at least one biodegradable LNP composition as described herein. In certain embodiments, the method further comprises co-administering a gene therapy vector with the biodegradable LNP composition.

In a further aspect, use of composition as described herein, is provided for delivering a gene product to a subject in need thereof.

In a further aspect, a composition as described herein is provided for delivering a gene product to a subject in need thereof.

In a further aspect, a method of treating or preventing at least one disease or disorder in a subject in need thereof is provided. The method includes administering a therapeutically effectively amount of a composition as described herein to the subject.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A is the structure of the biodegradable ionizable lipid termed B1.

FIG. 1B is the structure of the biodegradable ionizable lipid termed B3.

FIG. 2A is a schematic of the formulation strategy for the BLNPs described herein.

FIG. 2B is a table showing characteristics of the LNPs incorporating benchmark (C12-200), B1 or B3 lipids.

FIG. 2C is a graph showing luminescence intensity of benchmark (C12-200), B1, or B3 lipids in an in vitro screen.

FIG. 3A through FIG. 3D show data generated using B3 BLNPs that facilitated nanoparticle uptake, EGFP mRNA transfection and endosomal escape comparing with benchmark (C12-200) in vitro. FIG. 3A depicts LNP uptake and EGFP expression on Hela cells treated by C12-200 (Top) and B3 (Bottom) LNPs carrying EGFP mRNA. Biodegradable B3 LNPs showed higher cellular uptake and stronger EGFP expression. DiD fluorescence dye was used to label LNPs at a concentration of 0.2%. Samples were incubated for 3 h before imaging. Scale bar: 20 μm. FIG. 3B depicts a representative qualification of LNPs uptake by measuring DiD intensity from flow cytometry. B3 exhibited much higher DiD intensity than C12-200. FIG. 3C depicts a representative qualification of EGFP-LNPs expression by measuring EGFP intensity from flow cytometry. B3 exhibited much higher EGFP transfection efficiency than C12-200. FIG. 3D depicts representative endosomal escape of luciferase mRNA-loaded C12-200 (Top) and B3 (Bottom) LNPs. Hela cells were treated with 0.5 g/mL luciferase mRNA encapsulated in LNPs as indicated for 3 h. DiO was used to label the LNPs at a concentration of 1%. Lysotracker was used to stain the endosome for 1 h, while Hoechst were used to stain the nucleus for 5 min. Samples and dye markers were washed off before imaging. B3 LNPs treated cells displayed weaker overlapping of green and red colors than C12-200 LNPs, demonstrating enhanced endosomal escape capability. Scale bar: 20 μm.

FIG. 4 depicts the results of in vivo evaluation of B1 and B3 BLNPs and C12-200 at a luciferase mRNA dose (0.1 mg/kg). Bioluminescence images of whole bodies and various organs were recorded 12 h after i.v. injection of LNPs into C57BL/6 mice. B1 and B3 BLNPs showed similar or higher mRNA transfection in vivo compared with C12-200 in whole body and liver.

FIG. 5A and FIG. 5B depict a liver toxicity assay after injection of LNPs encapsulating luciferase-encoding mRNA. FIG. 5A depicts a representative alanine transaminase (ALT) quantification (±standard deviation) for control, BLNPs (B1 and B3 LNPs), and benchmark (C12-200 LNPs). n=5 biological animals. FIG. 5B depicts a representative aspartate transaminase (AST) quantification (±standard deviation) for control, two representative BLNPs (B1 and B3 LNPs), and benchmark (C12-200 LNPs). n=5 biological animals. C57BL/6J mice were dosed with 1.0 mg/kg luciferase mRNA LNPs, and liver enzymes were quantified 12 h after injection. B1 and B3 BLNPs showed much lower liver toxicity than benchmark. Statistical significance was calculated using Multiple t test with unpaired design. ***P<0.001; **P<0.01.

FIG. 6 shows percent GFP-positive cells at 160 hours post treatment with LNP (1-control; 2-C12-200 Cas9/gRNA=4-1 0.2 μg/mL; 3-C12-200 Cas9/gRNA-4-1 0.4 μg/mL; 4-C12-200 Cas9/gRNA=4-1 0.6 μg/mL; 5-C12-200 Cas9/gRNA=4-1 2 μg/mL; 6-B1 Cas9/gRNA=4-1 0.2 μg/mL; 7-B1 Cas9/gRNA=4-1 0.4 μg/mL; 8-B1 Cas9/gRNA=4-1 0.6 μg/mL; 9-B1 Cas9/gRNA=3-1 2 μg/mL; 10-B3 Cas9/gRNA=4-1 0.2 μg/mL; 11-B3 Cas9/gRNA=4-1 0.4 μg/mL; 12-B3 Cas9/gRNA=4-1 0.6 μg/mL; 13-B3 Cas9/gRNA=3-1 2 μg/mL).

FIG. 7A shows schematic representation of the study.

FIG. 7B shows summary of the analysis for on-target DNA editing, plotted as average percent of indel frequency.

FIG. 7C shows summary of the analysis for TTR protein reduction, plotted as average percent of serum TTR reduction.

FIG. 8A further shows summary of the analysis for on-target DNA editing, plotted as average percent of indel frequency, as compared with the LNP formulations composing C12-490, S5 and S7 lipid.

FIG. 8B shows summary of the analysis for TTR protein reduction, plotted as average percent of serum TTR reduction, as compared with the LNP formulations composing C12-490, S5 and S7 lipid.

FIG. 9A shows a schematic overview of the study design for examining the dose-dependent response for B3-LNP formulated CRISPR/Cas9 editing. FIG. 9B demonstrates gene editing efficiency by B3 is dependent on the dose and only mildly reduced in the absence of LDL receptor. The graph shows the indel frequency for systemic administration of 0, 0.2, 0.5, 1.0, and 2.0 mg RNA/kg in C57Bl/6J mice and 1.0 mg RNA/kg in LDLR/ApoB1—deficient mice. Mice were euthanized at 7 days post infusion. B3 LNP characteristics are also shown.

FIG. 9C shows the results of systemic administration of TTR CRISPR LNP at 2.0 mg RNA/kg, determined at 7 days post IV. Liver editing at this dosage is approximately 50%.

FIG. 10A shows a schematic overview of PK/PD study design for evaluating B3 LNP encapsulating CRISPR/Cas9 components. FIG. 10B is a graph demonstrating that B3 LNPs demonstrate rapid kinetics for gene editing and cargo clearance. 1.0 mg RNA/kg were administered IV to mice, that were then sacrificed 4, 24, 96 and 168 hours post IV. FIG. 10C shows in situ hybridization of Cas9 mRNA at the listed time points. Cas9 mRNA is shown in red, and nuclear (DAPI) staining is shown in blue. Cas9 mRNA decreases significantly throughout the time course.

FIG. 11A shows a schematic overview of a study design for evaluating LNP toxicity in Sprague Dawley rats (n=4 per group, 2 male (2M) and 2 female (2F); 6-8 week-old).

FIGS. 11B-11D show that systemic administration of B3 LNP in Sprague-Dawley rats is well tolerated. Body weights, ALTs, and ASTs of PBS- and B3 LNP-treated (2.0 mg RNA/kg) rats are shown.

FIG. 12 shows a schematic overview of a study design in a PCSK9-hE7 mouse line having a humanized Pcsk9 gene using Arcus2 or Cas9 mRNA.

FIG. 13 shows a schematic overview of a study design for evaluation of hPCSK9 sgRNA in PCSK9-hE7 mice.

FIG. 14 shows a schematic overview for lipid nanoparticle (LNP) encapsulation of Cas9 mRNA and gene targeting sgRNA.

FIGS. 15A and 15B show the results of a purification study as described in Example 4. LNPs encapsulating Cas9 mRNA and mTTR sgRNA were formulated using the NanoAssemblr® Blaze™ (Precision Nanosystems) and either: 1) dialyzed against 1×PBS in dialysis cassettes and concentrated using Amicon centrifugal filters; or 2) processed using tangential flow filtration (TFF) to exchange buffer solution and concentrate LNPs. The resulting LNPs were characterized and injected to mice. Serum TTR levels in mice were measured at days −1 and 7. FIG. 15A shows that LNP size was comparable between the two methods. FIG. 15B shows that serum TTR levels were also consistent using either method.

FIG. 16 shows that mTTR efficacy was similar even after storing the LNP in fridge for 7 days.

FIG. 17 shows the results of the study described in Example 7. C57BL/6J mice were injected intravenously (I.V.; via lateral tail vein injection) with LNP comprising C12-200 or B3 lipid, Cas9 mRNA/TTR sgRNA (4:1) at a dose of 1.0 mg RNA per kg body weight. On D 7 serum samples were collected and analyzed for gene-editing kinetics by ELISA (i.e., serum TTR levels). FIG. 17 demonstrates gene editing efficiency of B3 is similar to the C12-200 benchmark.

### DETAILED DESCRIPTION

The present disclosure is based, in part, on the unexpected discovery of biodegradable lipidic compounds having the structure of Formula (Ia) and (Ib) that are shown to target liver. The compounds are useful for, inter alia, inclusion within LNPs that can be used for the delivery of nucleic acids, such as editing enzymes.

In one aspect, the present disclosure provides a lipid nanoparticle (LNP) that is biodegradable comprising at least one compound of Formula (Ia) or (Ib), and having encapsulated therein a coding sequence for an editing enzyme. In various embodiments, the LNP comprises one or more compounds of Formula (Ia) or (Ib) in a concentration range of about 0.1 mol % to about 99 mol %.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In one embodiment, upon in vivo administration, a prodrug is chemically converted to the biologically, pharmaceutically, or therapeutically active form of the compound. In another embodiment, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the disclosure is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

In certain embodiments, “pseudouridine” refers, in another embodiment, to m¹acp³Ψ (1-methyl-3-(3-amino-3-carboxypropyl) pseudouridine. In another embodiment, the term refers to m¹Ψ (1-methylpseudouridine). In another embodiment, the term refers to Ψm (2′-O-methylpseudouridine. In another embodiment, the term refers to m⁵D (5-methyldihydrouridine). In another embodiment, the term refers to m³Ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present disclosure.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “pharmacological composition,” “therapeutic composition,” “therapeutic formulation” or “pharmaceutically acceptable formulation” can mean, but is in no way limited to, a composition or formulation that allows for the effective distribution of an agent provided by the disclosure, which is in a form suitable for administration to the physical location most suitable for their desired activity, e.g., systemic administration. The pharmaceutical composition facilitates administration of the compound to an organism. Multiple techniques of administering a compound exist in the art including, but not limited to, intravenous, oral, aerosol, parenteral, ophthalmic, pulmonary and topical administration.

Non-limiting examples of agents suitable for formulation with the, e.g., compositions provided by the instant disclosure include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “therapeutic” as used herein means a treatment and/or prophylaxis. A therapeutic effect is obtained by suppression, diminution, remission, or eradication of at least one sign or symptom of a disease or disorder state.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present disclosure include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

An “effective amount” or “therapeutically effective amount”, as used herein, means an amount which provides a therapeutic or prophylactic benefit. An “effective amount” or “therapeutically effective amount” of a compound is that amount of compound which is sufficient to provide a beneficial effect to the subject to which the compound is administered. For example, a “therapeutically effective amount” of the LNPs is the amount that is sufficient or effective to prevent or treat (delay or prevent the onset of, prevent the progression of, inhibit, decrease or reverse) a disease or condition, including alleviating symptoms of such diseases.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

To “treat” a disease as the term is used herein, means to reduce the frequency or severity of at least one sign or symptom of a disease or disorder experienced by a subject.

As used herein, “treating a disease or disorder” means reducing the frequency with which a symptom of the disease or disorder is experienced by a patient. Disease and disorder are used interchangeably herein.

A disease or disorder is “alleviated” if the severity of a sign or symptom of the disease or disorder, the frequency with which such a sign or symptom is experienced by a patient, or both, is reduced.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Parenteral” administration of a composition includes, e.g., subcutaneous (s.c.), intravenous (i.v.), intramuscular (i.m.), or intrasternal injection, or infusion techniques.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### DESCRIPTION

Provided herein, in one aspect, are biodegradable lipidic compounds having the structure of Formula (IA) and (IB) that are useful in LNPs for targeting liver cells. These compounds are referred to herein as B1 (Formula (IA)) and B3 (Formula (IB)). In one aspect, a lipid nanoparticle (LNP) is provided that is biodegradable comprising at least the biodegradable lipid compound of B1. In another aspect, a LNP is provided that is biodegradable comprising at least the biodegradable lipid compound of B3.

In certain embodiments, the biodegradable lipid nanoparticles (LNP) are useful for delivering a nucleic acid to a liver cell, and are formed from: (a) at least one ionizable lipid compound having the structure of Formula (IA), Formula (IB), or combinations thereof, (b) at least one neutral phospholipid, (c) at least one cholesterol lipid, and (d) at least one polyethylene glycol (PEG) lipid. The LNP additionally includes (e) at least one nucleic acid encapsulated in the LNP. In certain embodiments, the nucleic acid is a coding sequence for an editing enzyme.

### Biodegradable Lipidic Compounds and Lipid Nanoparticles (LNP)

The present disclosure relates, in part, to compositions comprising biodegradable lipid nanoparticles (BLNPs) that include a lipidic compound that is biodegradable. In various embodiments, the lipidic compound is a compound having the structure of Formula (IA) (also referred to as B1):

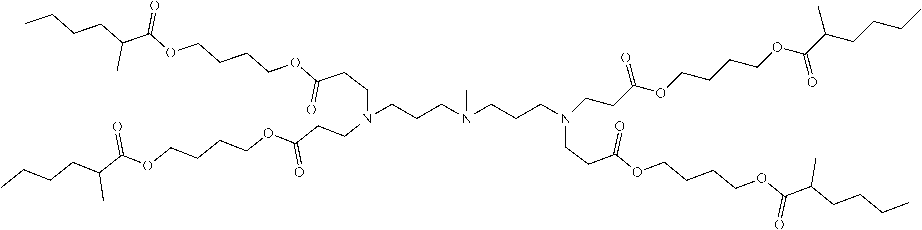 [SMILES: CCCCC(C)C(=O)OCCCCOC(=O)CCN(CCCN(C)CCCN(CCC(=O)OCCCCOC(=O)C(C)CCCC)CCC(=O)OCCCCOC(=O)C(C)CCCC)CCC(=O)OCCCCOC(=O)C(C)CCCC]

In various embodiments, the lipidic compound is a compound having the structure of Formula (IB) (also referred to as B3):

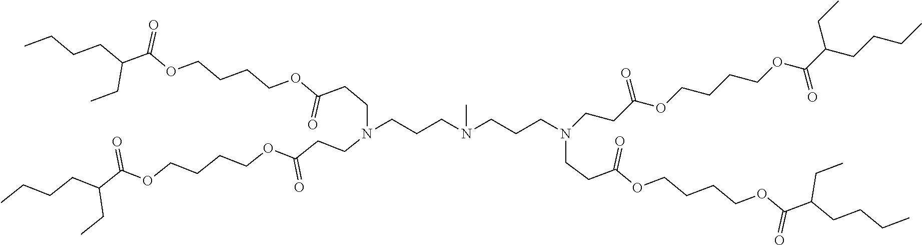 [SMILES: CCCCC(CC)C(=O)OCCCCOC(=O)CCN(CCCN(C)CCCN(CCC(=O)OCCCCOC(=O)C(CC)CCCC)CCC(=O)OCCCCOC(=O)C(CC)CCCC)CCC(=O)OCCCCOC(=O)C(CC)CCCC]

The present disclosure relates, in part, to a biodegradable lipid nanoparticle (LNP) comprising at least one biodegradable lipidic compound of B1 or B3 (e.g., a compound having the structure of Formula (IA) or (IB)) that is useful for targeting liver. In various embodiments, the LNP comprises one or more of B1 and/or B3 in a concentration range of about 0.1 mol % to about 99 mol %. In some embodiments, the LNP comprises one or more of B1 and/or B3 in a concentration range of about 1 mol % to about 95 mol %. In some embodiments, the LNP comprises one or more of B1 and/or B3 in a concentration range of about 10 mol % to about 70 mol %. In some embodiments, the LNP comprises one or more of B1 and/or B3 in a concentration range of about 10 mol % to about 50 mol %. In some embodiments, the LNP comprises one or more of B1 and/or B3 in a concentration range of about 15 mol % to about 45 mol %. In some embodiments, the LNP comprises one or more of B1 and/or B3 in a concentration range of about 35 mol % to about 40 mol %.

For example, in some embodiments, the LNP comprises one or more of B1 and/or B3 in a concentration of about 1 mol %. In some embodiments, the LNP comprises one or more of B1 and/or B3 in a concentration of about 2 mol %. In some embodiments, the LNP comprises one or more of B1 and/or B3 in a concentration of about 5 mol %. In some embodiments, the LNP comprises one or more of B1 and/or B3 in a concentration of about 5.5 mol %. In some embodiments, the LNP comprises one or more of B1 and/or B3 in a concentration of about 10 mol %. In some embodiments, the LNP comprises one or more of B1 and/or B3 in a concentration of about 12 mol %. In some embodiments, the LNP comprises one or more of B1 and/or B3 in a concentration of about 15 mol %. In some embodiments, the LNP comprises one or more of B1 and/or B3 in a concentration of about 20 mol %. In some embodiments, the LNP comprises one or more of B1 and/or B3 in a concentration of about 25 mol %. In some embodiments, the LNP comprises one or more of B1 and/or B3 in a concentration of about 30 mol %. In some embodiments, the LNP comprises one or more of B1 and/or B3 in a concentration of about 30 mol %. In some embodiments, the LNP comprises one or more of B1 and/or B3 in a concentration of about 31 mol %. In some embodiments, the LNP comprises one or more of B1 and/or B3 in a concentration of about 32 mol %. In some embodiments, the LNP comprises one or more of B1 and/or B3 in a concentration of about 33 mol %. In some embodiments, the LNP comprises one or more of B1 and/or B3 in a concentration of about 34 mol %. In some embodiments, the LNP comprises one or more of B1 and/or B3 in a concentration of about 35 mol %. In some embodiments, the LNP comprises one or more of B1 and/or B3 in a concentration of about 36 mol %. In some embodiments, the LNP comprises one or more of B1 and/or B3 in a concentration of about 37 mol %. In some embodiments, the LNP comprises one or more of B1 and/or B3 in a concentration of about 38 mol %. In some embodiments, the LNP comprises one or more of B1 and/or B3 in a concentration of about 39 mol %. In some embodiments, the LNP comprises one or more of B1 and/or B3 in a concentration of about 40 mol %. In some embodiments, the LNP comprises one or more of B1 and/or B3 in a concentration of about 41 mol %. In some embodiments, the LNP comprises one or more of B1 and/or B3 in a concentration of about 42 mol %. In some embodiments, the LNP comprises one or more of B1 and/or B3 in a concentration of about 43 mol %. In some embodiments, the LNP comprises one or more of B1 and/or B3 in a concentration of about 44 mol %. In some embodiments, the LNP comprises one or more of B1 and/or B3 in a concentration of about 45 mol %. In some embodiments, the LNP comprises one or more of B1 and/or B3 in a concentration of about 46 mol %. In some embodiments, the LNP comprises one or more of B1 and/or B3 in a concentration of about 47 mol %. In some embodiments, the LNP comprises one or more of B1 and/or B3 in a concentration of about 48 mol %. In some embodiments, the LNP comprises one or more of B1 and/or B3 in a concentration of about 49 mol %. In some embodiments, the LNP comprises one or more of B1 and/or B3 in a concentration of about 50 mol %. In some embodiments, the LNP comprises one or more of B1 and/or B3 in a concentration of about 55 mol %. In some embodiments, the LNP comprises one or more of B1 and/or B3 in a concentration of about 60 mol %. In some embodiments, the LNP comprises one or more of B1 and/or B3 in a concentration of about 70 mol %. In some embodiments, the LNP comprises one or more of B1 and/or B3 in a concentration of about 80 mol %. In some embodiments, the LNP comprises one or more of B1 and/or B3 in a concentration of about 90 mol %. In some embodiments, the LNP comprises one or more of B1 and/or B3 in a concentration of about 95 mol %. In some embodiments, the LNP comprises one or more of B1 and/or B3 in a concentration of about 95.5 mol %. In some embodiments, the LNP comprises one or more of B1 and/or B3 in a concentration of about 99 mol %.

In certain embodiments, the compound of B1 or B3 comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP. In certain embodiments, the compound of B1 or B3 comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP. In certain embodiments, the compound of Bl or B3 comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP. In certain embodiments, the LNP includes the ionizable lipid of B1, a phospholipid, a cholesterol lipid, and a PEG lipid. In some embodiments, the LNP includes the ionizable lipid of B3, a phospholipid, a cholesterol lipid, and a PEG lipid. The phospholipid, cholesterol lipid, and/or PEG lipid is sometimes referred to as a helper lipid.

The LNP includes a neutral phospholipid. In some embodiments, the phospholipid is dioleoyl-phosphatidylethanolamine (DOPE) or a derivative thereof, dioleoylphosphatidylcholine (DOPC) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, stearoyloleoylphosphatidylcholine (SOPC) or a derivative thereof, N-(2,3-dioleoyloxy) propyl)-N,N,N-trimethylammonium chloride (DOTAP) or a derivative thereof, DATAP or a derivative thereof, or any combination thereof. In certain embodiments, the phospholipid is DOPE.

For example, in some embodiments, the LNP comprises a phospholipid in a concentration range of about 5 mol % to about 45 mol %. In some embodiments, the LNP comprises a phospholipid in a concentration range of about 6 mol % to about 25 mol %. In some embodiments, the LNP comprises a phospholipid in a concentration range of about 6 mol % to about 12 mol %. In some embodiments, the LNP comprises a phospholipid in a concentration range of about 8 mol % to about 12 mol %. In some embodiments, the LNP comprises a phospholipid in a concentration of about 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, or 45 mol %. In some embodiments, the LNP comprises DOPE in a concentration of about 4 mol %. In some embodiments, the LNP comprises DOPE in a concentration of about 10 mol %. In some embodiments, the LNP comprises DOPE in a concentration of about 16 mol %. In some embodiments, the LNP comprises DOPE in a concentration of about 22 mol %. In some embodiments, the LNP comprises DOPE in a concentration of about 28 mol %.

The LNP includes a cholesterol lipid. In some embodiments, the cholesterol lipid is cholesterol or a derivative thereof, such as a substituted cholesterol molecule. In some embodiments, the LNP comprises a mixture of cholesterol and a substituted cholesterol molecule. For example, in some embodiments, the LNP comprises total cholesterol lipid including cholesterol and one or more substituted cholesterol in a concentration range of about 1 mol % to about 99 mol %. In some embodiments, the LNP comprises a total cholesterol lipid in a concentration range of about 5 mol % to about 75 mol %. In some embodiments, the LNP comprises total cholesterol lipid in a concentration range of about 5 mol % to about 55 mol %. In some embodiments, the LNP comprises total cholesterol lipid in a concentration range of about 20 mol % to about 50 mol %. In some embodiments, the LNP comprises total cholesterol lipid in a concentration range of about 40 mol % to about 55 mol %. In some embodiments, the LNP comprises a total cholesterol lipid in a concentration of about 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol %. In some embodiments, the LNP comprises total cholesterol lipid in a concentration of about 29.5 mol %. In some embodiments, the LNP comprises total cholesterol lipid in a concentration of about 28.5 mol %. In some embodiments, the LNP comprises total cholesterol lipid in a concentration of about 35 mol %. In some embodiments, the LNP comprises total cholesterol lipid in a concentration of about 39.5 mol %. In some embodiments, the LNP comprises total cholesterol lipid in a concentration of about 46.5 mol %. In some embodiments, the LNP comprises total cholesterol lipid in a concentration of about 51 mol %. In some embodiments, the LNP comprises total cholesterol lipid in a concentration of about 51.5 mol %. In some embodiments, the LNP comprises total cholesterol lipid in a concentration of about 53.5 mol %.

The LNP includes a polyethylene glycol (PEG) lipid. Examples of such PEG lipids include, but are not limited to, 1,2-dimyristoyl-sn-glycero-3-phosphoethanolamine-N-[methoxy (polyethylene glycol)-2000] (C14-PEG2000) or a derivative thereof, 1,2-dimyristoyl-rac-glycero-3-methoxypolyethylene glycol-2000 (DMG-PEG2000) or a derivative, and/or 1,2-distearoyl-sn-glycero-3-phosphoethanolamine-N-[amino (polyethylene glycol)-2000] (DSPE-PEG 2000 amine) or a derivative. For example, in some embodiments, the LNP comprises a polymer in a concentration range of about 0.1 mol % to about 25 mol %. In some embodiments, the LNP comprises a polymer in a concentration range of about 0.5 mol % to about 12.5 mol %. In some embodiments, the LNP comprises a polymer in a concentration range of about 0.5 mol % to about 3.5 mol %. In some embodiments, the LNP comprises a polymer in a concentration range of about 0.5 mol % to about 2.5 mol %. In some embodiments, the LNP comprises a polymer in a concentration range of about 1.0 mol % to about 2.5 mol %. In some embodiments, the LNP comprises a polymer in a concentration about 0.5 mol %. In some embodiments, the LNP comprises a polymer in a concentration about 1.0 mol %. In some embodiments, the LNP comprises a polymer in a concentration about 1.5 mol %. In some embodiments, the LNP comprises a polymer in a concentration about 2.0 mol %. In some embodiments, the LNP comprises a polymer in a concentration about 2.5 mol %. In some embodiments, the LNP comprises a polymer in a concentration about 3.0 mol %. In some embodiments, the LNP comprises a polymer in a concentration about 3.5 mol %.

In various embodiments, the LNP of the present disclosure comprises at least one compound having the structure of Formula (IA) or (IB), phospholipid, total cholesterol, and PEG-lipid, wherein the at least one compound having the structure of Formula (IA) or (IB): phospholipid:total cholesterol:PEG-lipid are present in a molar ratio of about 1-80:5-45:5-55:0. 5-12.5 or at a molar percentage of about 1-80%:5-45%:5-55%:0.5-12.5%. In one embodiment, the LNP comprises at least one compound having the structure of Formula (IA) or (IB), phospholipid, total cholesterol and PEG-lipid, wherein the at least one compound having the structure of Formula (IA) or (IB): phospholipid:total cholesterol:PEG-lipid are present in a molar ratio of about 35-45:5-20:40-55:1-2.5 or at a molar percentage of about 35-45%:5-20%:40-55%:1-2.5%. In one embodiment, the LNP comprises at least one compound having the structure of Formula (IA) or (IB), phospholipid, total cholesterol and PEG-lipid, wherein the at least one compound having the structure of Formula (IA) or (IB): phospholipid:total cholesterol:PEG-lipid are present in a molar ratio of about 30-35:16:46.5:2.5 or at a molar percentage of about 35%:16%:46.5%:2.5%. In one embodiment, the LNP comprises at least one compound having the structure of Formula (IA) or (IB), phospholipid, total cholesterol and PEG-lipid, wherein the at least one compound having the structure of Formula (IA) or (IB): phospholipid:total cholesterol:PEG-lipid are present in a molar ratio of about 35:16:46.5:2.5 or at a molar percentage of about 30-35%:16%:46.5%:2.5%.

In certain embodiments, the LNP comprises 35 mol % of a compound of B1, 16 mol % of a phospholipid, 46.5 mol % of a cholesterol lipid, and 2.5 mol % of a PEG-lipid. In certain embodiments, the LNP comprises 35 mol % of a compound of B1, 16 mol % of a DOPE, 46.5 mol % of cholesterol, and 2.5 mol % of C14PEG2000.

In certain embodiments, the LNP comprises 35 mol % of a compound of B3, 16 mol % of a phospholipid, 46.5 mol % of a cholesterol lipid, and 2.5 mol % of a PEG-lipid. In certain embodiments, the LNP comprises 35 mol % of a compound of B3, 16 mol % of a DOPE, 46.5 mol % of cholesterol, and 2.5 mol % of C14PEG2000.

Other exemplary molar ratios of the LNP components are found in Table 1 below. The column “ionizable lipid” refers to a compound of B1 or B3.

TABLE 1
Exemplary LNP Formulations

| Ionizable Lipid (a) | Phospholipid (b) | Cholesterol (c) | PEG-Lipid (d) |
|---|---|---|---|
| 40 | 4 | 53.5 | 2.5 |
| 60 | 10 | 29.5 | 0.5 |
| 40 | 28 | 28.5 | 3.5 |
| 40 | 28 | 29.5 | 2.5 |
| 40 | 22 | 35.0 | 3.0 |
| 35 | 22 | 40.0 | 3.0 |
| 35 | 22 | 39.5 | 3.5 |
| 30 | 16 | 51.0 | 3.0 |
| 30 | 16 | 51.5 | 2.5 |
| 40 | 16 | 42.5 | 1.5 |
| 35 | 16 | 46.5 | 2.5 |

The term “lipid nanoparticle” refers to a particle having at least one dimension on the order of nanometers (e.g., 1-1,000 nm) which includes one or more lipids, for example a lipid of Formula (Ia) or (Ib). In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the biodegradable LNP has a diameter of between about 50 nm to about 500 nm. In some embodiments, the biodegradable LNP has a diameter of between about 50 nm to about 160 nm.

In various embodiments, the lipids or the LNP of the present disclosure are substantially non-toxic.

The compositions provided herein are useful for targeting liver. In certain embodiments, the compositions are useful for gene editing applications. Thus, in certain embodiments, the LNPs provided herein include a coding sequence for an editing enzyme encapsulated therein. This coding sequence is sometimes referred to as a cargo.

In certain embodiments, the LNP compositions provided herein comprise at least one nucleic acid sequence encoding an editing enzyme or a transcript therefor. In certain embodiments, the compositions comprising gene editing enzymes exclude any nucleic acids encoding products for gene therapy and/or gene replacement. Optionally, the LNP compositions are co-administered with a viral vector (e.g., AAV) comprising a gene therapy and/or gene replacement.

Editing enzymes include various types of nucleases that are used to cut nucleic acid molecules. Such enzymes include zinc finger nucleases, Transcription activator-like effector nucleases (TALENs), meganucleases, clustered regularly interspaced short palindromic repeats (CRISPR) associated protein (CAS, e.g., CAS9), OMEGA enzymes (IscB), etc.

In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., CAS9 nuclease, a meganuclease that has been engineered to bind to site different than the cognate binding site). In other embodiments, the nuclease comprises heterologous DNA-binding and cleavage domains (e.g., zinc finger nucleases; TAL-effector nucleases; meganuclease DNA-binding domains with heterologous cleavage domains).

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms and serve as a prominent tool in the field of genome editing. In certain embodiments, the coding sequence encodes a zinc finger.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). In another embodiment, the coding sequence encodes a transcription activator-like (TAL) effector nuclease (TALEN).

In certain embodiments, the coding sequence encodes a CRISPR-associated nuclease (Cas9). “Cas9” (CRISPR associated protein 9) refers to family of RNA-guided DNA endonucleases which is characterized by two signature nuclease domains, RuvC (cleaves non-coding strand) and HNH (coding strand). Suitable bacterial sources of Cas9 include Staphylococcus aureus (SaCas9), Streptoococcus pyogenes (SpCas9), and Neisseria meningitides (K M Estelt et al, Nat Meth, 10:1116-1121 (2013), incorporated herein by reference). The wild-type coding sequences may be utilized in the constructs described herein. Alternatively, the bacterial codons are optimized for expression in humans, e.g., using any of a variety of known human codon optimizing algorithms. Alternatively, these sequences may be produced synthetically, either in full or in part. Other endonucleases with similar properties may optionally be substituted. See, e.g., the public CRISPR database (db) accessible at http://crispr.u-psud.fr/enspr.

In certain embodiments, the coding sequence encodes a meganuclease. Meganucleases are endodeoxyribonucleases characterized by a large recognition site (double-stranded DNA sequences of 12 to 40 base pairs), for example, I-SceI. When combined with a nuclease, DNA can be cut at a specific location. The restriction enzymes can be introduced into cells, for use in gene editing or for genome editing in situ. In certain embodiments, the nuclease is a member of the LAGLIDADG (SEQ ID NO: 3) family of homing endonucleases. In certain embodiments, the nuclease is a member of the I-CreI family of homing endonucleases which recognizes and cuts a 22 base pair recognition sequence SEQ ID NO: 4-CAAAACGTCGTGAGACAGTTTG. See, e.g., WO 2009/059195. Methods for rationally-designing mono-LAGLIDADG homing endonucleases were described which are capable of comprehensively redesigning I-CreI and other homing endonucleases to target widely-divergent DNA sites, including sites in mammalian, yeast, plant, bacterial, and viral genomes (WO 2007/047859). The term “homing endonuclease” is synonymous with the term “meganuclease.” See, WO 2018/195449, describing certain PCSK9 meganucleases, which is incorporated herein in its entirety.

In certain embodiments, the compositions described herein include coding sequences for editing enzymes, particularly nucleases, which are useful targeting a gene for the insertion of a transgene. In certain situations, for example, for applications that do not require precise targeting of an existing gene or locus (e.g., to introduce or modify an endogenous gene, allele, or regulatory element), a common strategy is to target transgene integration to one of a small number of genomic “safe harbor” sites (SHS) for expression, presumably without disrupting the expression of adjacent or more distant genes. These putative SHS play an increasingly important role in developing effective gene therapies; in the investigation of gene structure, function, and regulation; and in cell-based biotechnology.

Certain SHS are known in the art, or may be discovered. Known SHS include the AAVS1 site on chromosome 19q, CCR5 chemokine receptor gene, ROSA26, PCKS9, and albumin (Alb). See, e.g., Monnat et al, New Human Chromosomal Sites with “Safe Harbor” Potential for Targeted Transgene Insertion, Hum Gene Ther. 2019 Jul. 1; 30 (7): 814-828, which is incorporated by reference. In certain embodiments, the editing enzyme targets a SHS.

In certain embodiments, the editing enzyme is a nuclease that is specific for Proprotein convertase subtilisin/kexin type 9 (PCSK9). In other embodiments, the editing enzyme is a nuclease that is specific for albumin (Alb). See, e.g., Conway et al, Non-viral Delivery of Zinc Finger Nuclease mRNA Enables Highly Efficient In Vivo Genome Editing of Multiple Therapeutic Gene Targets, Molecular Therapy, 27(4):866-877 (April 2019), which is incorporated herein by reference. In one embodiment, the nuclease is a meganuclease such as that described, e.g., in International Patent Publication No. WO 2018/195449.

In some embodiments, the LNP further includes sequences which direct the nuclease to a target site in the target locus. As used herein, the term “target site” or “target sequence” refers to the specific nucleotide sequence that is recognized by the editing enzyme, or its guide sequence. The “target locus” or “target gene locus” is any site in the gene coding region where insertion of the heterologous transgene is desired. For example, in certain embodiments, the target PCSK9 locus is in Exon 7 of the PCSK9 coding sequence located on chromosome 1.

In certain embodiments, such as a meganuclease specific for PCSK9, TTR, or albumin, no further sequences are required to direct the nuclease to the target site. However, in the case, for example, of Cas9, an additional sequence, called a “single guide RNA” or “sgRNA” is provided, which is specific for the target sequence. As used herein, the sgRNA has at least a 20-base sequence (or about 24-28 bases, sometimes called the seed region) for specific DNA binding (i.e., homologous to the target DNA), in combination with the gRNA scaffold. Transcription of sgRNAs should start precisely at the 5′ end. When targeting the template DNA strand, the base-pairing region of the sgRNA has the same sequence identity as the transcribed sequence. When targeting the non-template DNA strand, the base-pairing region of the sgRNA is the reverse-complement of the transcribed sequence. Optionally, the LNP may contain more than one sgRNA. The sgRNA is 5′ to a protospacer-adjacent motif (PAM) which is specifically recognized by the Cas9 (or Cpf1) enzyme. Typically, the sgRNA is “immediately” 5′ to the PAM sequence, i.e., there are no spacer or intervening sequences. Suitable sgRNAs can be designed by the person of skill in the art.

The sgRNA includes at least 20 nucleotides and specifically binds to a target site in the target gene, said target site being 5′ to a protospacer-adjacent motif (PAM) that is specifically recognized by the Cas9. The seed region in some embodiments shares 100% complementarity with the target site in the target gene. In other embodiments, the seed region contains 1, 2, 3, 4, or 5 mismatches as compared to the target site.

In other embodiments, for example, wherein the nuclease is a Cas9, the gene editing vector further includes one or more nuclear localization signal (NLSs). In one embodiment, the NLSs flank the coding sequence for the Cas9. See, e.g., Lu et al. Types of nuclear localization signals and mechanisms of protein import into the nucleus, Cell Commun Signal (May 2021) 19:60.

In certain embodiments, the cargo is a DNA molecule or an RNA molecule. In certain embodiments, the cargo is a cDNA or mRNA molecule. In various embodiments, the composition comprises an in vitro transcribed (IVT) RNA molecule. For example, in certain embodiments, the composition comprises an IVT RNA molecule, which encodes an editing enzyme. In certain embodiments, the IVT RNA molecule is a nucleoside-modified mRNA molecule. In certain embodiments, where the nuclease coding sequence is provided as messenger RNA (mRNA). An mRNA may include a 5′ untranslated region, a 3′ untranslated region, and/or a coding or translating sequence.

An mRNA may be a naturally or non-naturally occurring mRNA. An mRNA may include one or more modified nucleobases, nucleosides, or nucleotides. In some embodiments, the mRNA in the compositions comprise at least one modification which confers increased or enhanced stability to the nucleic acid, including, for example, improved resistance to nuclease digestion in vivo. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present disclosure, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. Also contemplated by the terms “modification” and “modified” as such terms related to the mRNA of the present disclosure are alterations which improve or enhance translation of mRNA nucleic acids, including for example, the inclusion of sequences which function in the initiation of protein translation (e.g., the Kozak consensus sequence).

In some embodiments, the mRNA described herein have undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids of the present disclosure also include the incorporation of pseudouridines pseudouridine (ψ) or 5-methylcytosine (m5C). Substitutions and modifications to the mRNA of the present disclosure may be performed by methods readily known to one or ordinary skill in the art.

In certain embodiments, the mRNA includes a 5′ cap structure, a chain terminating nucleotide, a stem loop, a polyA sequence, and/or a polyadenylation signal. A cap structure or cap species is a compound including two nucleoside moieties joined by a linker and may be selected from a naturally occurring cap, a non-naturally occurring cap or cap analog, or an anti-reverse cap analog. An mRNA may instead or additionally include a chain terminating nucleoside.

In certain embodiments, the mRNA includes a stem loop, such as a histone stem loop. A stem loop may include 1, 2, 3, 4, 5, 6, 7, 8, or more nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

In certain embodiments, the mRNA includes a poly A sequence. A polyA sequence may be comprised entirely or mostly of adenine nucleotides or analogs or derivatives thereof. In certain embodiments, the poly A sequence is a tail located adjacent to a 3′ untranslated region of an mRNA.

In one embodiment, the present disclosure provides a method for gene editing of a liver cell of interest of a subject (e.g., a liver cell). For example, the method can be used to provide one or more component of a gene editing system (e.g., a component of a CRISPR system) to a cell of interest of a subject. In certain embodiments, a second nucleic acid molecule is provided that encodes a transgene of interest, or an expression cassette containing the transgene coding sequence. In certain embodiments, the transgene is provided as mRNA. In other embodiments, the transgene is provided as DNA

In certain embodiments, the LNP compositions provided herein comprise at least one nucleic acid sequence encoding a gene having therapeutic effect, e.g., for gene replacement or to correct a disorder or deficiency. In certain embodiments, the LNPs comprising a therapeutic gene exclude any gene editing enzymes.

In some embodiments, the transgene is a therapeutic agent. In certain embodiments, the transgene relates to a liver metabolic disorder. In certain embodiments, the transgene is OTC, PKU, CTLN1, or LDLR.

In certain embodiments, the transgene encodes a protein that is aberrantly expressed in a liver metabolic disorder or other genetic disorder. In certain embodiments, the transgene encodes a protein other than PCSK9. Such proteins include, but are not limited to OTC, low density lipoprotein receptor (LDLr), Factor IX, and. Factor VIII.

Further illustrative genes which may be delivered via the compositions described herein include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); low density lipoprotein receptor (LDLR) protein, associated with familial hypercholesterolemia (FH), LDLR variant, such as those described in WO 2015/164778; ApoE and ApoC proteins, associated with dementia; lipoprotein lipase (LPL) (Lipoprotein Lipase Deficiency), UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GMI gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; carnosinase (CN1); hypoxanthine-guanine phosphoribosyltransferase (HGPRT); erythropoietin (EPO); Carbamyl Phosphate Synthetase (CPS1), N-Acetylglutamate Synthetase (NAGS); Argininosuccinate Lyase (ASL) (Argininosuccinic Aciduria); and Arginase (AG); argininosuccsinate synthase associated with adult onset type II citrullinemia (CTLN2) (WO 2018/144709, which is incorporated herein by reference); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

In certain embodiments, the indicated hepatic diseases, disorders, syndrome and/or conditions include, but not limited to, liver disease (associated with hydroxysteroid 17-beta dehydrogenase 13 (HSD17B13) encoding gene, non-alcoholic steatohepatitis (NASH) (associated with diacylglycerol O-acyltransferase-2 (DGAT2), hydroxysteroid 17-Beta Dehydrogenase 13 (HSD17B13), or patatin-like phospholipase domain-containing 3 (PNPLA3) encoding genes), and alcohol use disorder (associated with aldehyde dehydrogenase 2 (ALDH2) encoding gene).

In certain embodiments, the indicated endocrine or metabolic diseases, disorders, syndrome and/or conditions include, but not limited to, hypertriglyceridemia (associated with apolipoprotein C-III (APOC3), or angiopoietin-like 3 (ANGPTL3) encoding genes), lipodystrophy, hyperlipidemia (associated with apolipoprotein C-III (APOC3) encoding gene), hypercholesterolemia (associated with apolipoprotein B-100 (APOB-100), proprotein convertase subtilisin kexin type 9 (PCSK9)), or amyloidosis (associated with transthyretin (TTR) encoding gene), porphyria (associated with aminolevulinate synthase-1 (ALAS-1) encoding gene), neuropathy (associated with transthyretin (TTR) encoding gene), primary hyperoxaluria type 1 (associated with glycolate oxidase encoding gene), diabetes (associated with Glucagon receptor (GCGR) encoding gene), acromegaly (growth hormone receptor (GHR) encoding gene), alpha-1 antitrypsin deficiency (AATD) (associated with alpha-1 antitrypsin (AAT) encoding gene), propionic acidemia (propionyl-CoA carboxylase (PCCA/PCCB) encoding gene), glycogen storage disease type III (GDSIII) (associated with glycogen debranching enzyme (GSDIII) encoding gene), cardiometabolic disease (associated with asialoglycoprotein (ASGPR), hydroxyacid Oxidase 1 (HAO1), or alpha-1-antitrypsin (SERPINA1) encoding genes), methylmalonic acidemia (MMA) (associated with methylmalonyl CoA mutase (MMUT), cob (I) alamin adenosyltransferase (MMAA or MMAB), methylmalonyl-CoA epimerase (MCEE), LMBR1 domain containing 1 (LMBRD1), or ATP-binding cassette subfamily D member 4 (ABCD4) encoding genes), glycogen storage disease type 1a (associated with Glucose-6-phosphatase catalytic subunit-related protein (G6PC) encoding gene), and phenylketonuria (PKU) (associated with phenylalanine hydroxylase (PAH) encoding gene).

Examples of suitable transgenes for delivery include, e.g., those associated with familial hypercholesterolemia (e.g., VLDLr, LDLr, ApoE, see, e.g., WO 2020/132155, WO 2018/152485, WO 2017/100682, which are incorporated herein by reference), muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), progranulin (PRGN) (associated with non-Alzheimer's cerebral degenerations, including, frontotemporal dementia (FTD), progressive non-fluent aphasia (PNFA) and semantic dementia), among others. Other useful gene products include, carbamoyl synthetase I, ornithine transcarbamylase (OTC), arginosuccinate synthetase, arginosuccinate lyase (ASL) for treatment of arginosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). Examples of suitable transgene for delivery may include human frataxin delivered in an AAV vector as described, e.g., PCT/US20/66167, Dec. 18, 2020, U.S. Provisional Patent Application No. 62/950,834, filed Dec. 19, 2019, and U.S. Provisional Application No. 63/136,059 filed on Jan. 11, 2021 which are incorporated herein by reference. Another example of suitable transgene for delivery may include human acid-α-glucosidase (GAA) delivered in an AAV vector as described, e.g., PCT/US20/30493, Apr. 30, 2020, now published as WO2020/223362A1, PCT/US20/30484, Apr. 20, 2020, now published as WO 2020/223356 A1, U.S. Provisional Patent Application No. 62/840,911, filed Apr. 30, 2019, US Provisional Application No. 62/913,401, filed Oct. 10, 2019, U.S. Provisional Patent Application No. 63/024,941, filed May 14, 2020, and U.S. Provisional Patent Application No. 63/109,677, filed Nov. 4, 2020 which are incorporated herein by reference. Also, another example of suitable transgene for delivery may include human a-L-iduronidase (IDUA) delivered in an AAV vector as described, e.g., PCT/US2014/025509, Mar. 13, 2014, now published as WO 2014/151341, and U.S. Provisional Patent Application No. 61/788,724, filed Mar. 15, 2013 which are incorporated herein by reference.

In some embodiments, the therapeutic cargo is an siRNA, miRNA, shRNA, or an antisense molecule, which inhibits a targeted nucleic acid including those encoding proteins that are involved in aggravation of the pathological processes.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391 (19): 306-311; Timmons et al., 1998, Nature 395:854; Montgomery et al., 1998, TIG 14 (7): 255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216.

In one aspect, the disclosure includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic cargos. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a delivery vehicle of the disclosure. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the disclosure is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the disclosure or the gene construct of the disclosure can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

In certain embodiments of the disclosure, an antisense nucleic acid sequence, which is expressed by a plasmid vector is used as a therapeutic cargo to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the disclosure may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the disclosure include oligonucleotide derivatives known in the art which have improved biological activity compared to unmodified oligonucleotides (see U.S. Pat. No. 5,023,243).

In certain embodiments of the disclosure, a ribozyme is used as a therapeutic cargo to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available recargos (Applied Biosystems, Inc., Foster City, CA) or they may be genetically expressed from DNA encoding them.

In certain embodiments, the cargo comprises a miRNA or a mimic of a miRNA. In certain embodiments, the cargo comprises a nucleic acid molecule that encodes a miRNA or mimic of a miRNA.

miRNAs are small non-coding RNA molecules that are capable of causing post-transcriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA cargos featured in the disclosure can be synthesized in vivo by a cell-based system or in vitro by chemical synthesis.

In various embodiments, the cargo comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide cargos featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3 ‘-terminus can be blocked with an aminoalkyl group. Other 3’ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose and so forth) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and recargos of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another cargo, e.g., another therapeutic cargo or an cargo that stabilizes an oligonucleotide cargo, e.g., a protein that complexes with the oligonucleotide cargo. Still other cargos include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present disclosure may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present disclosure encompass oligomeric compound comprising oligonucleotides having a certain identity to any nucleobase sequence version of a miRNAs described herein. In certain embodiments, the transgene may be operably linked to regulatory sequences that direct the expression thereof. In some embodiments, the transgene cassette includes a promoter, the transgene coding sequence, and a poly A sequence. In some embodiments, the promoter is a liver-specific promoter, such as the TBG promoter, TBG-SI promoter, HLP promoter, or others known in the art. In other embodiments, a transgene is provided without a promoter, and is inserted in the genome downstream of a native promoter, e.g., the PCSK9 promoter.

In addition to a promoter, the transgene cassette may contain one or more appropriate “regulatory elements” or “regulatory sequences”, which comprise but are not limited to an enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha 1-microglobulin/bikunin enhancer), amongst others. These control sequences or the regulatory sequences are operably linked to the nuclease coding sequences or transgene coding sequence.

In addition to the transgene cassette, in certain embodiments, the LNP composition described herein also includes homology-directed recombination (HDR) arms 5′ and 3′ to the transgene cassette, to facilitate homology directed recombination of the transgene into the endogenous genome. The homology arms are directed to the target locus and can be of varying length. In some embodiments, the HDR arms are from about 100 bp to about 1000 bp in length. In other embodiments, the HDR arms are from about 130 bp to about 500 bp. In other embodiments, the HDR arms are from about 100 bp to about 300 bp. In one embodiment, the HDR arm is 130 bp. In other embodiments, the HDR arms are about 130 bp to 140 bp. In another embodiment, the HDR arms are about 500 bp. In another embodiment, the HDR arms are absent. The HDR arms ideally share a high level of complementarity with the target locus, although it need not be 100% complementarity. In some embodiments, 1, 2, 3, 4, 5, 6, 7, 8, 9, 10, 11, 12, 13, 14, 15, 16, 17, 18, 19, 20, or more mismatches are permitted in each HDR arm.

The ratio of ionizable lipid to nucleic acid may be varied in the LNP in a range from about 4:1 to about 10:1 by weight. In certain embodiments, the ionizable lipid:nucleic acid ratio is about 5:1. In certain embodiments, the ionizable lipid:nucleic acid ratio is about 6:1. In certain embodiments, the ionizable lipid:nucleic acid ratio is about 7:1. In certain embodiments, the ionizable lipid:nucleic acid ratio is about 8:1. In certain embodiments, the ionizable lipid:nucleic acid ratio is about 9:1. In certain embodiments, the ionizable lipid:nucleic acid ratio is about 10:1.

In some embodiments, the weight ratio of (a): the at least one nucleic acid is between about 1:1 to about 10:1. In some embodiments, the LNP comprises, or encapsulates, at least one nucleic acid.

In embodiments where the composition includes a coding sequence for a Cas9 (e.g., mRNA encoding Cas9), the mRNA to sgRNA ratio can be present in a range of from about 1:5 to about 5:1 by weight. In certain embodiments, the mRNA:sgRNA ratio is about 1:5. In certain embodiments, the mRNA:sgRNA ratio is about 1:4. In certain embodiments, the mRNA:sgRNA ratio is about 1:3. In certain embodiments, the mRNA:sgRNA ratio is about 1:2. In certain embodiments, the mRNA:sgRNA ratio is about 1:1. In certain embodiments, the mRNA:sgRNA ratio is about 2:1. In certain embodiments, the mRNA:sgRNA ratio is about 3:1. In certain embodiments, the mRNA:sgRNA ratio is about 4:1. In certain embodiments, the mRNA:sgRNA ratio is about 5:1. Other ratios within this range can be utilized.

LNP formation and encapsulation of cargo may be accomplished using techniques known in the art. See, e.g., Jeffs, et al (March 2005). A Scalable, Extrusion-Free Method for Efficient Liposomal Encapsulation of Plasmid DNA. Pharmaceutical Research, 22(3), 362-372, and Kulkarni et al, On the Formation and Morphology of Lipid Nanoparticles Containing Ionizable Cationic Lipids and siRNA, ACS Nano, 12:4787-4795 (April 2018) both of which are incorporated herein by reference. For example, in brief, LNP-mRNA compositions are generated by rapid mixing of lipids in ethanol with mRNA in aqueous buffer (pH 4.0), followed by dialysis to remove ethanol and to raise the pH to 7.4.

In one embodiment, the nucleic acid is a nucleoside-modified RNA. Thus, in one aspect, the composition comprises a nucleoside-modified RNA. Thus, in one embodiment, the nucleic acid is a nucleoside-modified RNA In one embodiment, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present disclosure is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Kariko et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Karikó et al., 2011, Nucleic Acids Research 39:e142). Administering HPLC-purified, pseudourine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy.

The present disclosure encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In one embodiment, the nucleoside-modified RNA of the disclosure is IVT RNA. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine. In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the modified nucleoside of the present disclosure is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m1A (1-methyladenosine); m2A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl) adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl) uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl) uridine)); mchm⁵U (5-(carboxyhydroxymethyl) uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present disclosure are modified (e.g., either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

### Treatment Methods

The present disclosure provides methods of delivering an nucleic acid to a liver cell of a target subject. In certain embodiments, the nucleic acid is an editing enzyme. In some embodiments, the composition includes a therapeutic nucleic acid for the treatment or prevention of a disease or disorder. Therefore, in some embodiments, the disclosure provides methods for diagnosing, treating, or preventing a disease or disorder comprising administering an effective amount of the LNP composition comprising one or more nucleic acids, as described herein.

In some embodiments, the disclosure relates to methods of treating or preventing liver diseases or disorders and diseases or disorders associated therewith in subjects in need thereof, the method comprising administering the LNP composition of the disclosure. Exemplary liver diseases or disorders that can be treated using the LNP compositions and methods of the disclosure include, but are not limited to, hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, transthyretin (TTR), proprotein convertase subtilisin/kexin type 9 (PCSK9)-based diseases or disorders, or any combination thereof. Further disorders include glycogen storage disease or deficiency type 1A (GSD1), PEPCK deficiency, CDKL5 deficiency, galactosemia, phenylketonuria (PKU), Primary Hyperoxaluria Type 1, Maple syrup urine disease, tyrosinemia type 1, methylmalonic acidemia, medium chain acetyl CoA deficiency, ornithine transcarbamylase deficiency, citrullinemia; lecithin-cholesterol acyltransferase (LCAT) deficiency, amethylmalonic acidemia (MMA), Niemann-Pick disease, propionic academia (PA); familial hypercholesterolemia (FH), dementia, Lipoprotein Lipase Deficiency, Crigler-Najjar disease, severe combined immunodeficiency disease, Gout and Lesch-Nyan syndrome, biotimidase deficiency, Fabry disease, GM1 gangliosidosis, Wilson's Disease, Gaucher disease type 2 and 3, Zellweger syndrome, metachromatic leukodystrophy, Krabbe disease, Pompe disease, Nieman Pick disease type A, Argininosuccinic Aciduria, adult onset type II citrullinemia, urea cycle disorders; Farber lipogranulomatosis, aspartyl-glucosaminuria, fucosidosis, alpha-mannosidosis, acute intermittent porphyria (AIP), alpha-1 antitrypsin deficiency (emphysema), anemia due to thalassemia or to renal failure, ischemic diseases, occluded blood vessels as seen in, for example, atherosclerosis, thrombosis, or embolisms, Parkinson's disease, congestive heart failure, muscular dystrophies, and diabetes.

In one embodiment, the method comprises administering a LNP composition of the disclosure comprising one or more nucleic acid molecules for treatment or prevention of a disease or disorder, such as those described herein. In one embodiment, the one or more nucleic acid molecules encode an editing enzyme and, optionally, a therapeutic nucleic acid for the treatment of the disease or disorder.

In one embodiment, the compositions of the disclosure can be administered in combination with one or more additional therapeutic nucleic acid, an adjuvant, or a combination thereof. For example, in one embodiment, the method comprises administering an LNP composition comprising a nucleic acid molecule encoding an editing enzyme for targeted administration to a liver cell and a second LNP comprising a nucleic acid molecule encoding a therapeutic nucleic acid. In one embodiment, the method comprises administering a single LNP composition comprising a nucleic acid molecule encoding an editing enzyme and a nucleic acid molecule encoding a therapeutic nucleic acid.

In certain embodiments, the method comprises administering the LNP of the disclosure comprising nucleoside-modified RNA, which provides stable expression of a nucleic acid encoded editing enzyme described herein to a liver cell.

Administration of the compositions of the disclosure in a method of treatment can be achieved in a number of different ways, using methods known in the art. In one embodiment, the method of the disclosure comprises systemic administration of the composition, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition. In one embodiment, the method comprises direct delivery to the liver.

It will be appreciated that the composition of the disclosure may be administered to a subject either alone, or in conjunction with another nucleic acid.

The therapeutic and prophylactic methods of the disclosure thus encompass the use of pharmaceutical compositions comprising at least one LNP composition comprising a nucleic acid (e.g., an mRNA molecule encoding an editing enzyme) described herein, to practice the methods of the disclosure. The pharmaceutical compositions useful for practicing the methods may be administered to deliver a dose of from 0.001 ng/kg and 100 mg/kg nucleic acid, e.g., mRNA. For example, in some embodiments, the pharmaceutical compositions useful for practicing the disclosure may be administered to deliver a dose of from 0.5 mg/kg and 5 mg/kg mRNA. in some embodiments, the pharmaceutical compositions useful for practicing the disclosure may be administered to deliver a dose of at least or about 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5 mg/kg mRNA. In one embodiment, the disclosure envisions administration of a dose which results in a concentration of the LNP compositions from 10 nM and 10 μM in a mammal.

Typically, dosages which may be administered in a method to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 μg to about 10 mg per kilogram of body weight of the mammal. More preferably, the dosage will vary from about 1 μg to about 5 mg per kilogram of body weight of the mammal. For example, in some embodiments, the dosage will vary from about 0.5 mg to about 5 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of a composition of the present disclosure may be performed by single administration or boosted by multiple administrations.

In one embodiment, a method comprising administering a combination of LNP compositions described herein is provided. In certain embodiments, the combination has an additive effect, wherein the overall effect of the administering the combination is approximately equal to the sum of the effects of administering each LNP composition. In other embodiments, the combination has a synergistic effect, wherein the overall effect of administering the combination is greater than the sum of the effects of administering each LNP composition.

In one aspect, the compositions described herein may be administered prophylactically (i.e., to prevent disease or disorder, e.g., a disease described herein) or therapeutically (i.e., to treat disease or disorder, such as a disease described herein), to subjects suffering from or at risk of (or susceptible to) developing the disease or disorder. Such subjects may be identified using standard clinical methods.

In the context of the present disclosure, prophylactic administration occurs prior to the manifestation of overt clinical symptoms of disease or disorder, such that the disease or disorder is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The composition of the disclosure can be useful in combination with therapeutic, anti-cancer, and/or radiotherapeutic nucleic acids that are known to be useful in treating the disorder or disease. Thus, the present disclosure provides a combination of the present LNP with therapeutic, anti-cancer, and/or radiotherapeutic nucleic acids for simultaneous, separate, or sequential administration. The composition of the disclosure and the other anticancer nucleic acid can act additively or synergistically.

The therapeutic nucleic acid, anti-cancer nucleic acid, and/or radiation therapy can be administered according to therapeutic protocols well known in the art. It will be apparent to those skilled in the art that the administration of the therapeutic nucleic acid, anti-cancer nucleic acid, and/or radiation therapy can be varied depending on the disease being treated and the known effects of the anti-cancer nucleic acid and/or radiation therapy on that disease. Also, in accordance with the knowledge of the skilled clinician, the therapeutic protocols (e.g., dosage amounts and times of administration) can be varied in view of the observed effects of the administered therapeutic nucleic acids (i.e., anti-neoplastic nucleic acid or radiation) on the patient, and in view of the observed responses of the disease to the administered therapeutic nucleic acids, and observed adverse effects.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful herein may be prepared, packaged, or sold in formulations suitable for intrahepatic, ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active nucleic acids.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intracisternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing nucleic acids. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

In certain embodiments, the pharmaceutical composition includes a sugar solution. For example, in certain embodiments, sucrose is included in the solution at a concentration of from about 100 nM to about 500 nM. In certain embodiments, the sucrose solution is included at a concentration of at least or about 100 nM. In certain embodiments, the sucrose solution is included at a concentration of at least or about 200 nM. In certain embodiments, the sucrose solution is included at a concentration of at least or about 300 nM. In certain embodiments, the sucrose solution is included at a concentration of at least or about 400 nM. In certain embodiments, the sucrose solution is included at a concentration of at least or about 500 nM.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing nucleic acids, wetting nucleic acids, or suspending nucleic acids described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing nucleic acids. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing nucleic acids, wetting nucleic acids, or suspending nucleic acids described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active nucleic acids; dispersing nucleic acids; inert diluents; granulating and disintegrating nucleic acids; binding nucleic acids; lubricating nucleic acids; sweetening nucleic acids; flavoring nucleic acids; coloring nucleic acids; preservatives; physiologically degradable compositions such as gelatin; aqueous vehicles and solvents; oily vehicles and solvents; suspending nucleic acids; dispersing or wetting nucleic acids; emulsifying nucleic acids, demulcents; buffers; salts; thickening nucleic acids; fillers; emulsifying nucleic acids; antioxidants; antibiotics; antifungal nucleic acids; stabilizing nucleic acids; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, PA), which is incorporated herein by reference.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present disclosure and practice the claimed methods. The following working examples therefore, specifically point out the preferred embodiments of the present disclosure, and are not to be construed as limiting in any way the remainder of the disclosure.

### Example 1: Biodegradable Lipidoids and Lipid Nanoparticles Facilitated Systemic mRNA Delivery in Vivo

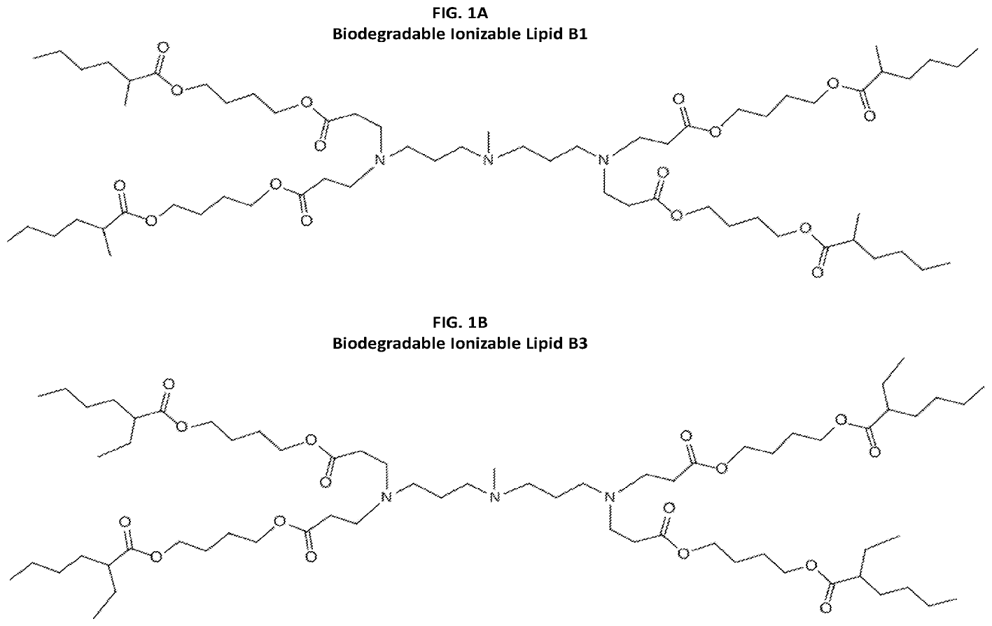

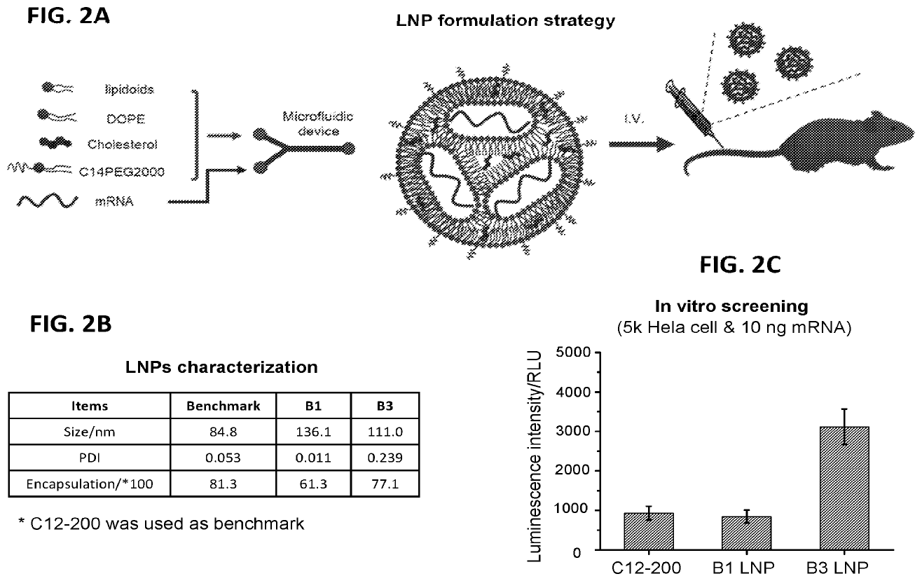

To develop novel LNP delivery systems with both high delivery efficacy and low toxicity, the present studies sought to identify degradable LNPs that enable potent mRNA expression in vivo than benchmark without causing off-target toxicities. As shown in FIG. 2A, biodegradable lipid nanoparticles (BLNPs) were formulated via microfluidic device with biodegradable ionizable lipids termed B1 (FIG. 1A) and B3 (FIG. 1B), helper lipid (DOPE), cholesterol, and PEG-lipid (C14PEG2000). The resulting B1 and B3 BLNPs were analyzed with Cryogenic transmission electron microscopy (cryo-TEM) for their size and structural analysis that demonstrated that the obtained BLNPs possessed a flower-like morphology (FIG. 2B and Data not shown).

Structure-activity relationships of B1 and B3 BLNPs were also accessed for luciferase mRNA delivery in vitro. Hela cells were treated with B1 and B3 BLNPs containing 10 ng mRNA. B1 LNPs showed luminescence similar to the C12-200 benchmark, while the B3 LNP showed about 3× the luminescence.

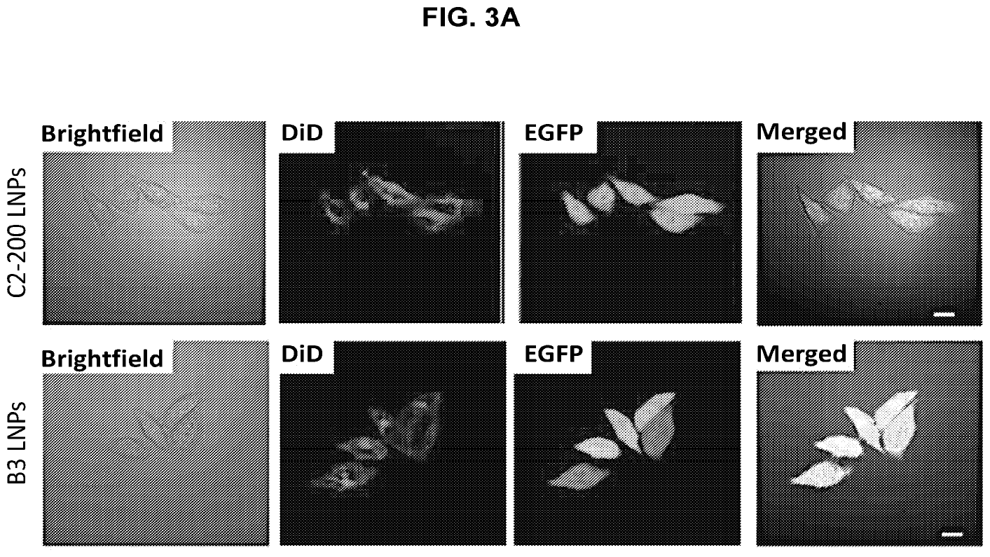

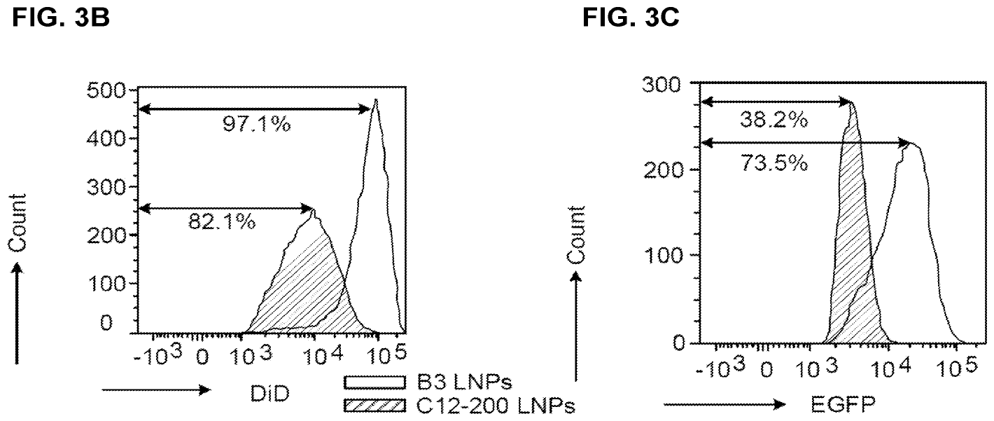

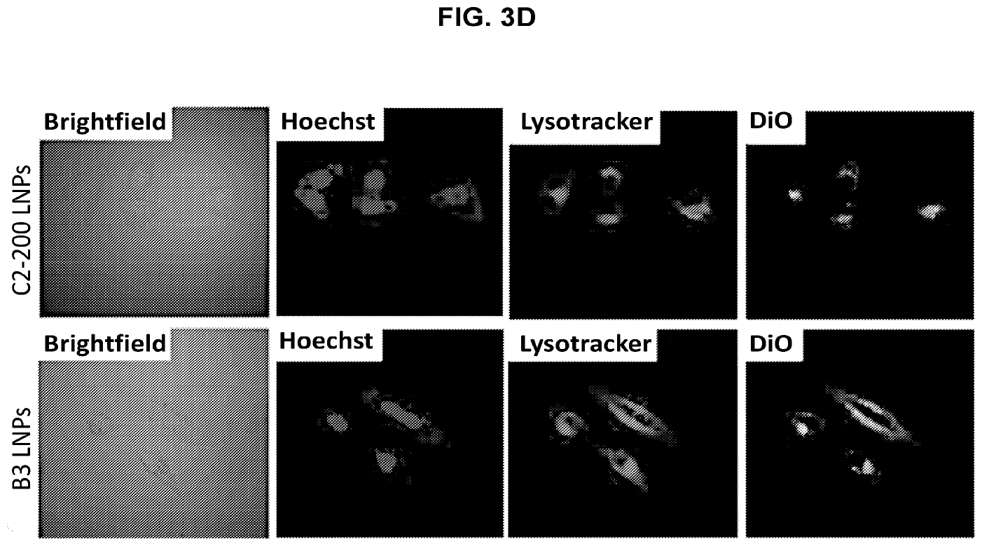

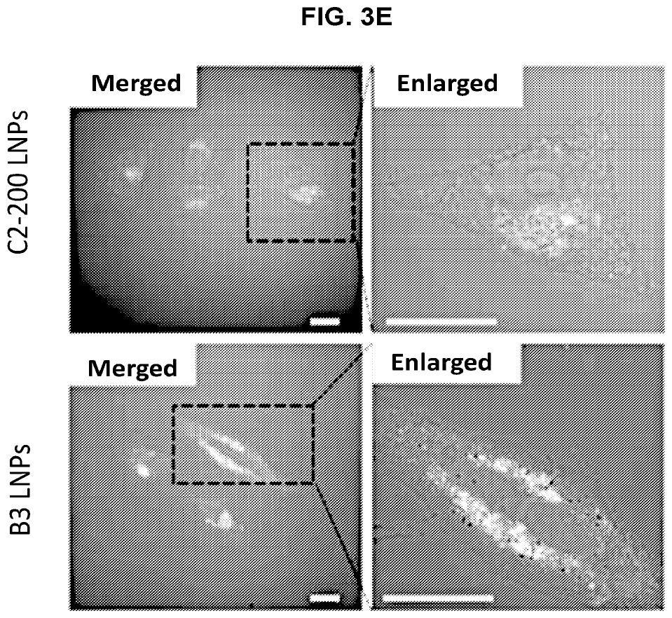

In addition, as shown in FIG. 3A-3D, the present studies also evaluated nanoparticle uptake, EGFP mRNA transfection, and endosomal escape facilitated by B3 BLNPs as compared to the benchmark C12-200 in vitro. Samples were incubated for 3 h before imaging and DiD fluorescence dye was used to label LNPs at a concentration of 0.2%. Biodegradable B3 LNPs showed higher cellular uptake and stronger EGFP expression (FIG. 3A). B3 also exhibited much higher DiD intensity than C12-200 (FIG. 3B) and exhibited much higher EGFP transfection efficiency than C12-200 (FIG. 3C). To evaluate endosomal escape capabilities, Hela cells were treated with 0.5 g/mL luciferase mRNA encapsulated in LNPs as indicated for 3 h. DiO was used to label the LNPs at a concentration of 1%. Lysotracker was used to stain the endosome for 1 h, while Hoechst were used to stain the nucleus for 5 min. Samples and dye markers were washed off before imaging (FIG. 3D). As shown in FIG. 3E, B3 LNPs treated cells displayed weaker overlapping of green and red colors than C12-200 LNPs, demonstrating enhanced endosomal escape capability.

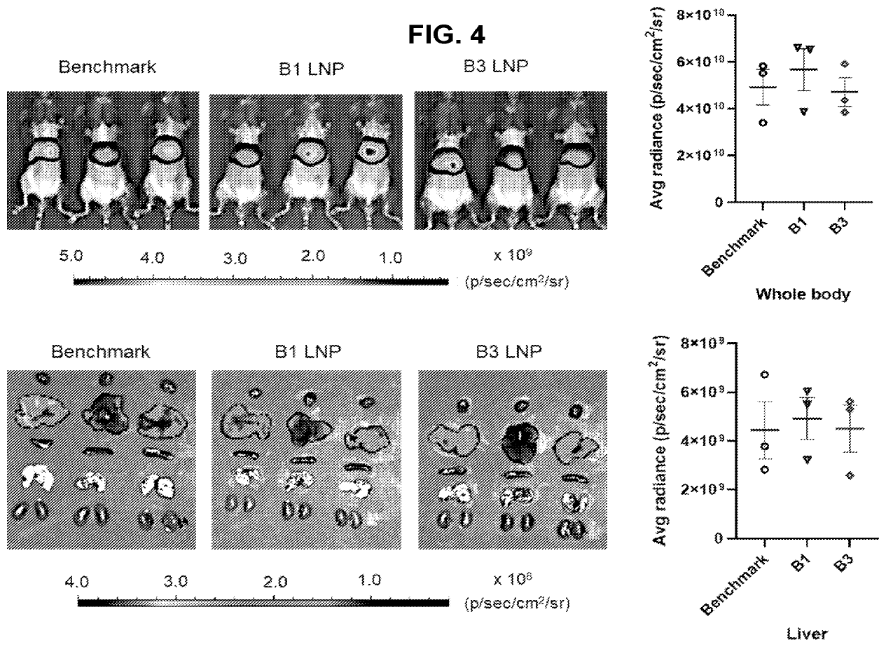

Subsequent structure-activity studies revealed that biodegradable lipid structure demonstrated significantly higher in vivo efficacy than the benchmark C12-200 (FIG. 4). More specifically, bioluminescence images of whole bodies and liver were recorded 12 h after i.v. injection of LNPs into C57BL/6 mice. B1 and B3 BLNPs showed higher mRNA transfection in vivo compared with C12-200 in liver (FIG. 4).

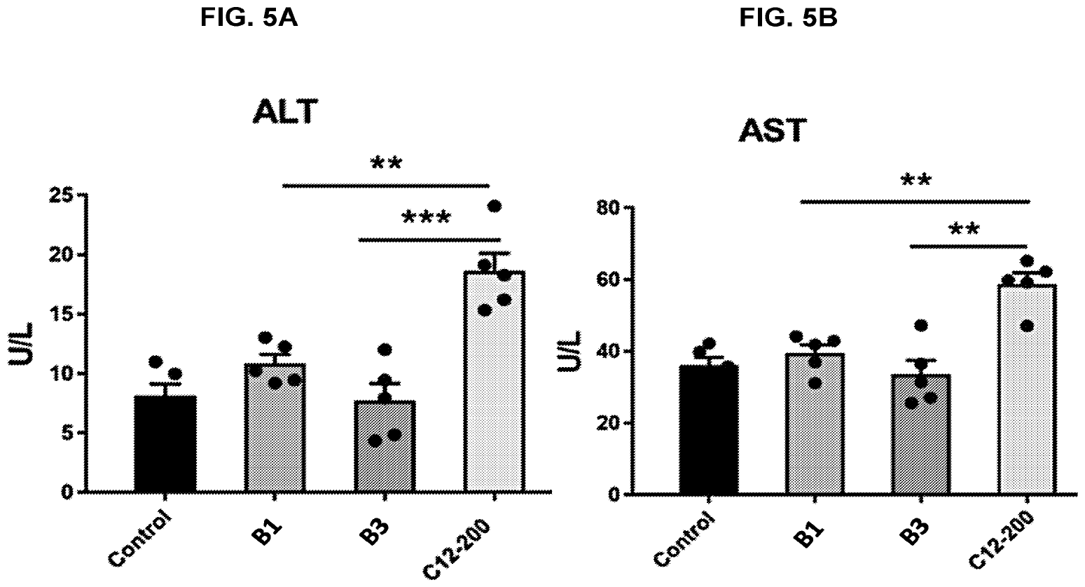

Lastly, a liver toxicity of BLNPs was assessed using a liver toxicity assay after injection of LNPs encapsulating luciferase-encoding mRNA. Alanine transaminase (ALT) quantification (±standard deviation) was used for control and compared to the BLNPs (B1 and B3 LNPs) and the benchmark C12-200 LNPs (FIG. 5A). In a separate study, aspartate transaminase (AST) quantification (±standard deviation) was for control and compared to two representative BLNPs (B1 and B3 LNPs) and the benchmark C12-200 LNPs (FIG. 5B). C57BL/6J mice were dosed with 1.0 mg/kg luciferase mRNA LNPs, and liver enzymes were quantified 12 h after injection. Two representative BLNPs showed much lower liver toxicity than benchmark.

In summary, the studies described herein provide novel biodegradable lipidoids and BLNP compositions that demonstrated high delivery efficacy and low toxicity for targeted delivery to liver.

### Example 2: In Vitro Gene Editing by Delivering Cas9 mRNA/sgRNA

Successful therapeutic application of CRISPR/Cas9 mediated genome editing requires a safe, organ specific, and efficacious delivery mechanism. There is growing utilization of ionizable lipid nanoparticles (LNPs) as a non-viral strategy for the in vivo delivery of RNA therapeutics. However, there remains a challenge to identify LNP formulations that provide potent but selective delivery of the RNA cargo. Here we report our efforts to identify novel ionizable lipids that confer robust delivery of intravenously administered LNP encapsulated RNA specifically to liver hepatocytes and the application of these LNP formulations for genome editing. We screened 29 LNP formulations comprised of novel ionizable lipids for liver-directed delivery of firefly luciferase mRNA upon systemic administration. Five candidate formulations emerged from this screen that resulted in potent luciferase expression in the liver but not in other highly perfused organs such as spleen, heart, kidney, and lung. These formulations produced LNPs that were well tolerated in mice and physically characterized by having a diameter ranging from 95 to 160 nm and a low polydispersity index. To test the efficacy of the new LNP formulations to deliver CRISPR/Cas9 components, we produced LNPs that were co-formulated with mRNA for S. py Cas9 and an sgRNA targeting the mouse transthyretin (TTR) gene. We varied the ratio of mRNA to sgRNA in the formulations in an attempt to optimize the formulation conditions to achieve maximum in vivo gene editing. We found that a subset of ionizable lipids initially identified in the luciferase mRNA screen could produce LNPs with efficacious delivery of CRISPR/Cas9 components to achieve clinically relevant levels of in vivo genome editing accompanied by a significant reduction of serum TTR protein. The degree of serum TTR reduction and on-target DNA indel formation was dependent on both the ionizable lipid present in the formulation and the mRNA to sgRNA ratio.

In this study, GFP-HepG2 cells (20, 000 cells) were treated with LNP comprising Trilink Cas9 mRNA/GFP for 160 hours. Various LNP were used: C12-200 LNP, B1 LNP and B3 LNP (see Example 1, above for further details). The summary of the LNP characterization is shown in the Table 2 below.

TABLE 2
LNP Characterization

| Items | Benchmark | B1 | B3 |
|---|---|---|---|
| Size/nm | 75.9 | 145.1 | 102.5 |
| PDI | 0.027 | 0.098 | 0.047 |
| Encapsulation/*100 | 87.9 | 63.2 | 75.1 |

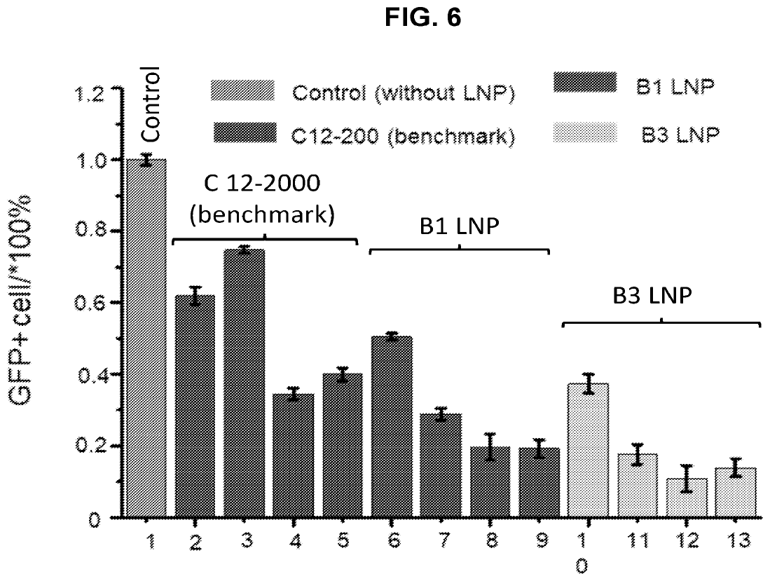

GFP-HepG2 cells were then analyzed for GFP expression. FIG. 6 shows percent GFP-positive cells at 160 hours post treatment with LNP (1-control; 2-C12-200 Cas9/gRNA=4-1 0.2 μg/mL; 3-C12-200 Cas9/gRNA=4-1 0.4 μg/mL; 4-C12-200 Cas9/gRNA-4-1 0.6 μg/mL; 5 C12-200 Cas9/gRNA=4-1 2 μg/mL; 6-B1 Cas9/gRNA-4-1 0.2 μg/mL; 7-B1 Cas9/gRNA=4-1 0.4 μg/mL; 8-B1 Cas9/gRNA=4-1 0.6 μg/mL; 9-B1 Cas9/gRNA-3-1 2 μg/mL; 10-B3 Cas9/gRNA=4-1 0.2 μg/mL; 11-B3 Cas9/gRNA=4-1 0.4 μg/mL; 12-B3 Cas9/gRNA=4-1 0.6 μg/mL; 13-B3 Cas9/gRNA-3-1 2 μg/mL). These results show improved efficiency in delivering mRNA/sgRNA to cells with B1-LNP and B3-LNP in comparison to C12-200 benchmark LNP, as observed in a significant decrease in percent GFP-positive cells.

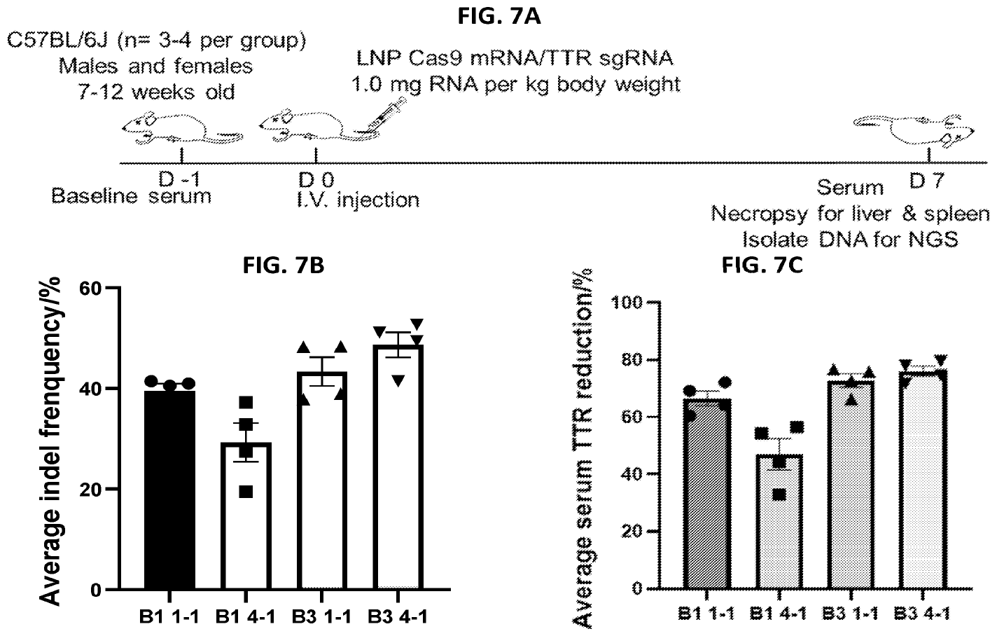

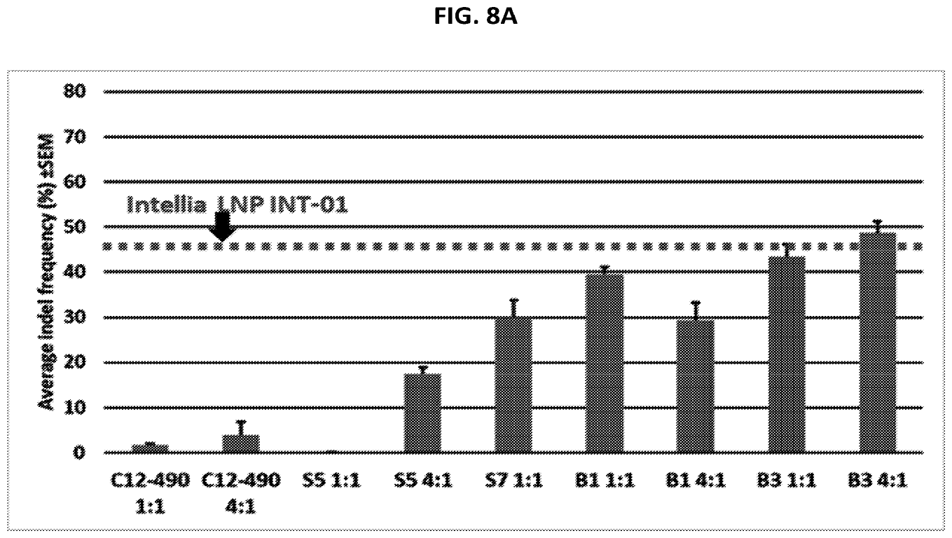

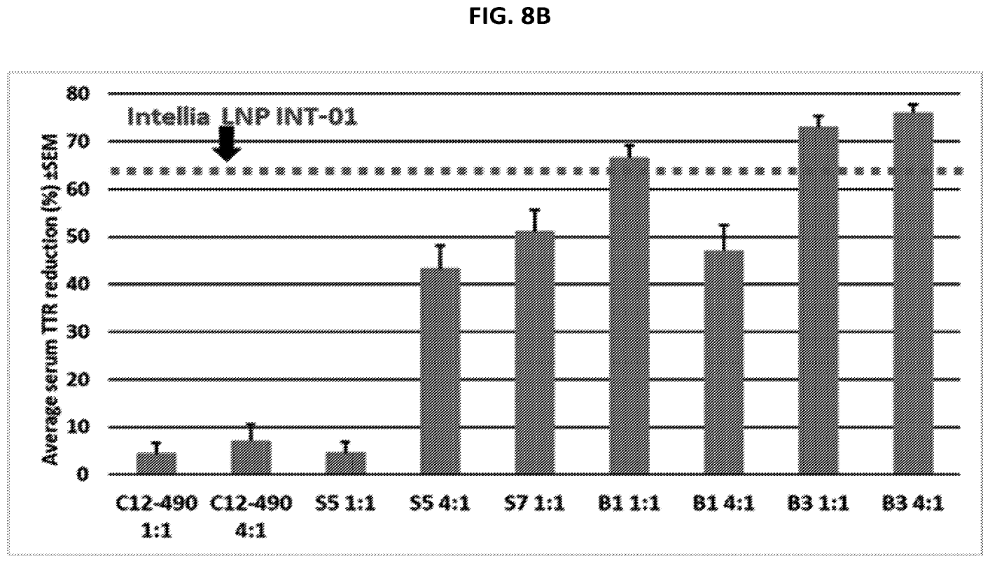

Next, we evaluated the LNP formulations for liver directed TTR editing in vivo (FIGS. 7A to 7C). In this study, C57BL/6J (n=3-4 per group; males and females; 7-12 weeks old) were used. On D −1 (i.e., 1 day prior to IV injection), baseline serum samples were collected for reference. On D 0, mice were injected intravenously (I.V.) with LNP comprising lipid (B1, B3, C12-490. S5, S7), Cas9 mRNA/TTR sgRNA at 1.0 mg RNA per kg body weight. LNPs were formulated at various ratios of mRNA:sgRNA, e.g., 1-1 and 4-1. On D 7 (i.e., 7-days post IV injection) serum samples were collected, mice were necropsied, and spleen tissue was collected for the purpose of DNA isolation for further NGS (sequencing) analysis. FIG. 7A shows schematic representation of the study. FIG. 7B shows summary of the analysis for on-target DNA editing, plotted as average percent of indel frequency. FIG. 7C shows summary of the analysis for TTR protein reduction, plotted as average percent of serum TTR reduction. FIG. 8A further shows summary of the analysis for on-target DNA editing, plotted as average percent of indel frequency, as compared with the LNP formulations composing C12-490, S5 and S7 lipid. FIG. 8B shows summary of the analysis for TTR protein reduction, plotted as average percent of serum TTR reduction, as compared with the LNP formulations composing C12-490, S5 and S7 lipid. These results show that mRNA/sgRNA ratio in B1-LNP, more specifically 4:1 instead of 1:1 (4-1, 1-1) yielded in the observed decrease in average percent indel frequency and in the observed average percent serum TTR reduction, while the ratio change to 4:1 in B3-LNP yielded in a slight increase. B3-LNP comprising mRNA/sgRNA at a ratio 4:1 was chosen for further experimentation.

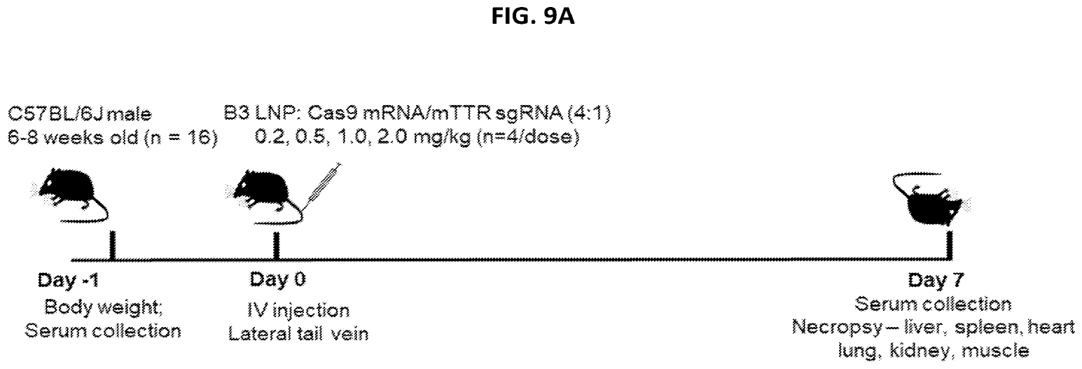

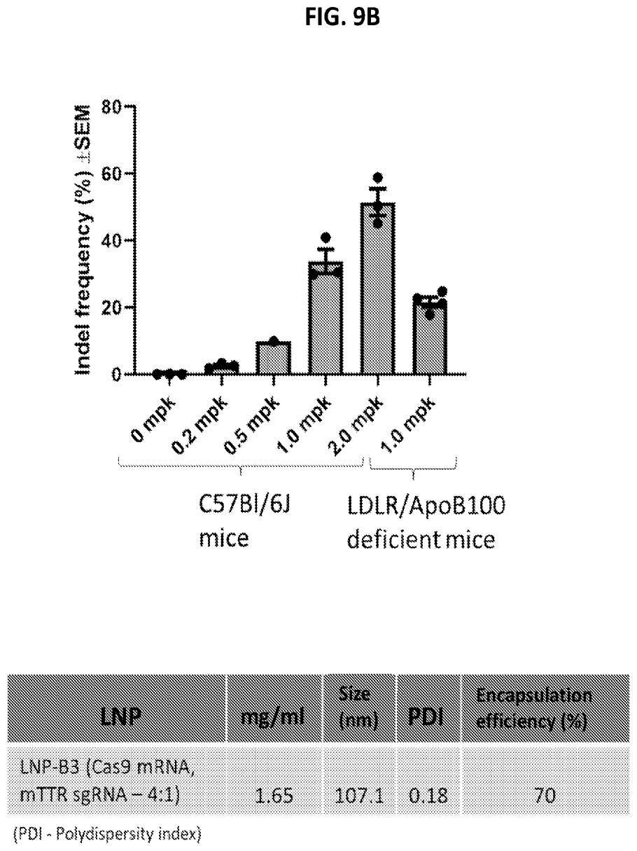

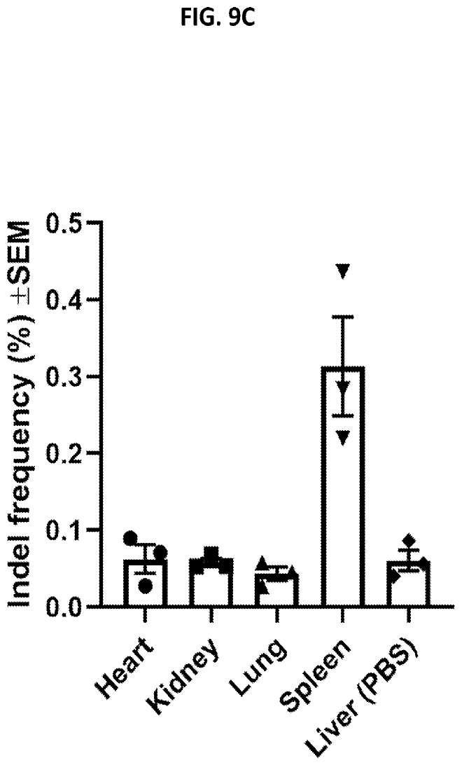

Further, we examined the dose response for the B3-LNP formulated CRISPR/Cas9 editing. FIG. 9A shows a schematic overview of the study design for examining the dose-dependent response for B3-LNP formulated CRISPR/Cas9 editing. In this study, C57BL/6J mice (male at 6-8 weeks old; n=16) were used. On D −1 (i.e., 1 day prior to IV injection), body weight and baseline serum samples were collected for reference. On D 0, mice were injected intravenously (I.V.; via lateral tail vein injection) with LNP comprising B3 lipid, Cas9 mRNA/TTR sgRNA (4:1) at doses of 0.2, 0.5, 1.0 and 2.0 mg RNA per kg body weight. On D 7 (i.e., 7-days post IV injection) body weight measurements were taken, mice were necropsied, serum samples were collected, and sample tissues were collected (spleen, heart, lung, kidney and muscle). Collected samples were analyzed for gene-editing kinetics by ELISA (i.e., serum TTR levels) and amplicon sequencing (i.e., DNA indel frequency). Additionally, the collected samples are subjected to toxicology analysis via liver histopathology and liver function tests (alanine aminotransferase (ALT), Aspartate aminotransferase (AST), alkaline phosphatase (Alk phos), and bilirubin). Additionally, collected samples are examined for RNA and protein expression kinetics using western blot for Cas9 from liver tissue lysates, RT-qPCR to detect Cas9 mRNA and sgRNA, in situ hybridization (ISH) to detect Cas9 mRNA, immunohistochemistry (IHC) to detect Cas9 protein. FIG. 9B demonstrates gene editing efficiency by B3 is dependent on the dose and only mildly reduced in the absence of LDL receptor. The graph shows the indel frequency for systemic administration of 0, 0.2, 0.5, 1.0, and 2.0 mg RNA/kg in C57Bl/6J mice and 1.0 mg RNA/kg in LDLR/ApoB1-deficient mice. As shown in FIG. 9B, gene editing efficiency was slightly reduced in the LDLR deficient mice

The tissues of the 2.0 mg RNA/kg group were analyzed for indel frequency as shown in FIG. 9C. At this dosage, liver editing is approximately 50%. These data, in conjunction with the data shown in FIG. 4A, show that mRNA delivery by an LNP does not accurately predict gene editing efficiency for the same LNP.

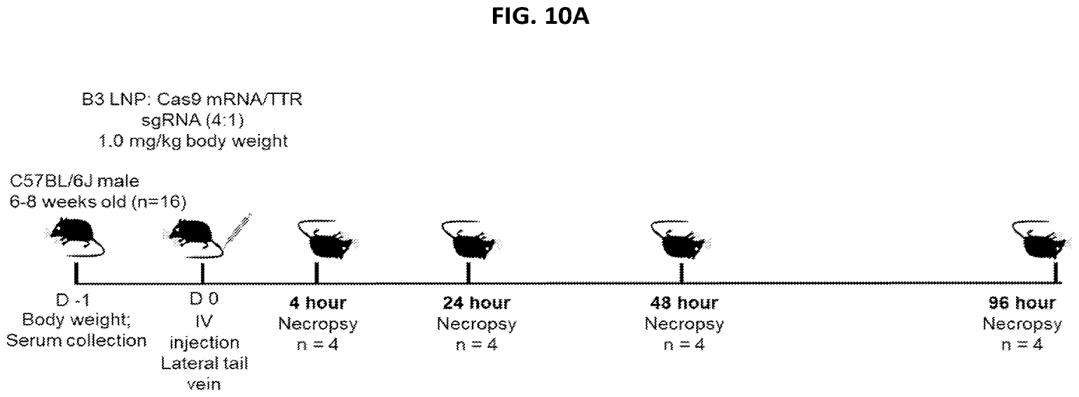

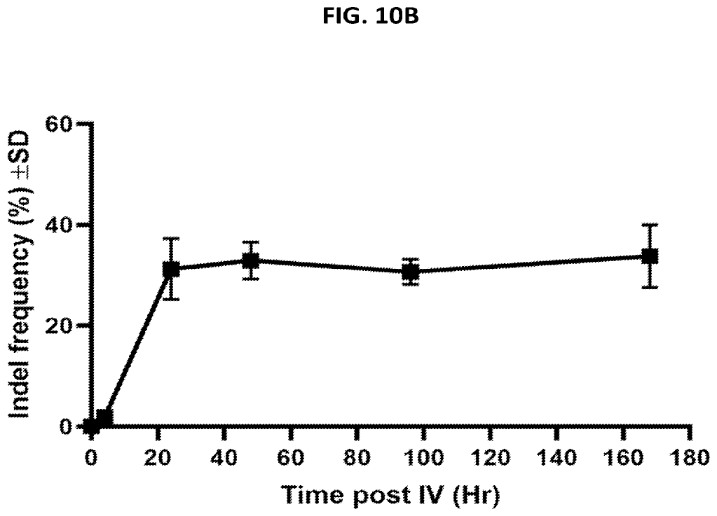

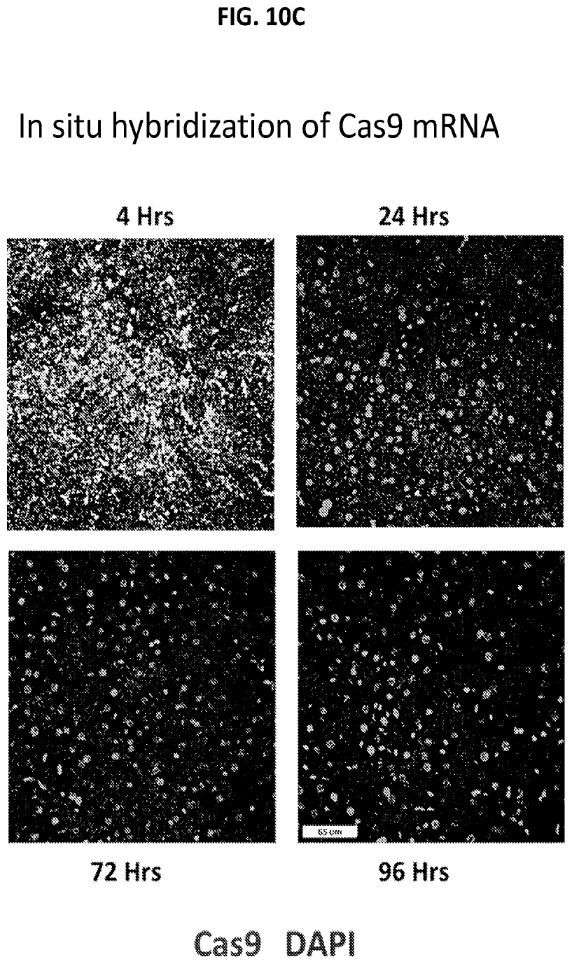

Next, we perform a pharmacokinetic-pharmacodynamic (PK/PD) study for B3 LNP encapsulating CRISPR/Cas9 components to examine in vivo effect. FIG. 10 shows a schematic overview of PK/PD study design for evaluating B3 LNP encapsulating CRISPR/Cas9 components. In this study, C57BL/6J mice (male at 6-8 weeks old; n=16) were used. On D −1 (i.e., 1 day prior to IV injection), body weight and baseline serum samples were collected for reference. On D 0, mice were injected intravenously (I.V.; via lateral tail vein injection) with LNP comprising B3 lipid, Cas9 mRNA/TTR sgRNA (4:1) at dose of 1.0 mg RNA per kg body weight. At 4-, 24-, 48-, and 96-hours post I. V. injection mice (n=4) are necropsied, and liver, spleen and blood (serum; via a cardiac puncture) were collected for analysis, as indicated in the study immediately above (i.e., gene editing kinetics, toxicology, and RNA and protein expression kinetics). FIG. 10B is a graph demonstrating that B3 LNPs demonstrate rapid kinetics for gene editing and cargo clearance. FIG. 10C shows in situ hybridization of Cas9 mRNA at 4, 24, 27, and 96 hours post dosage.

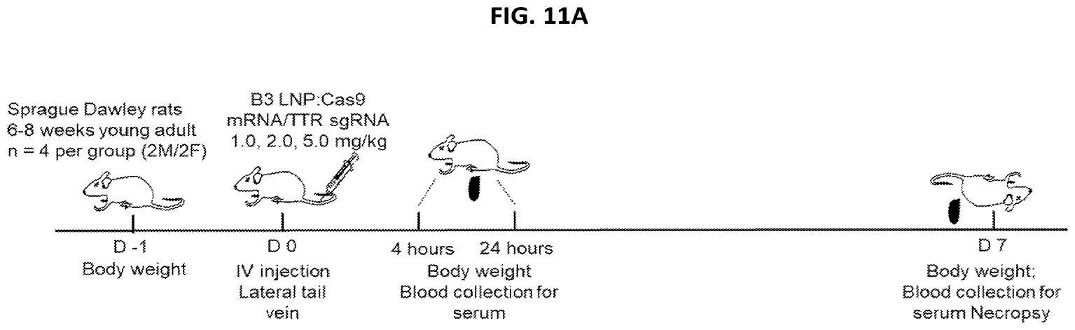

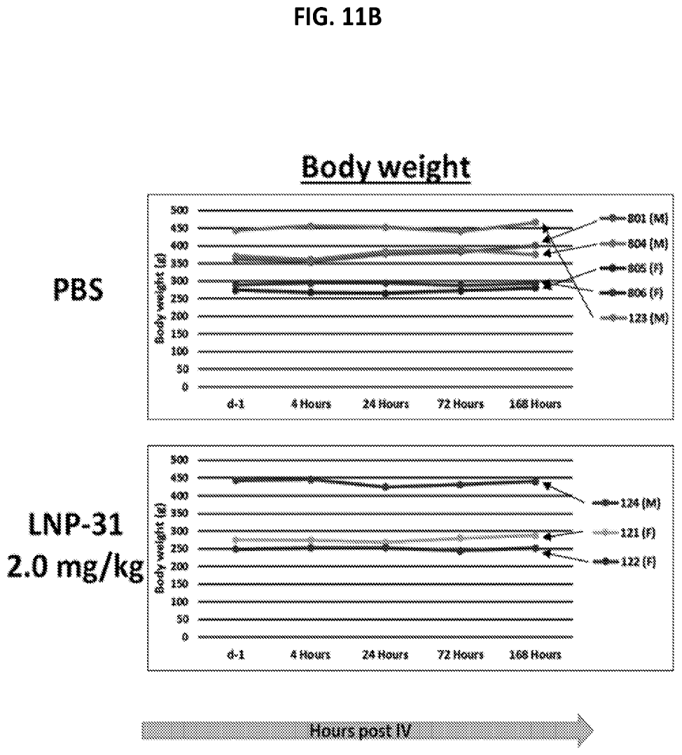

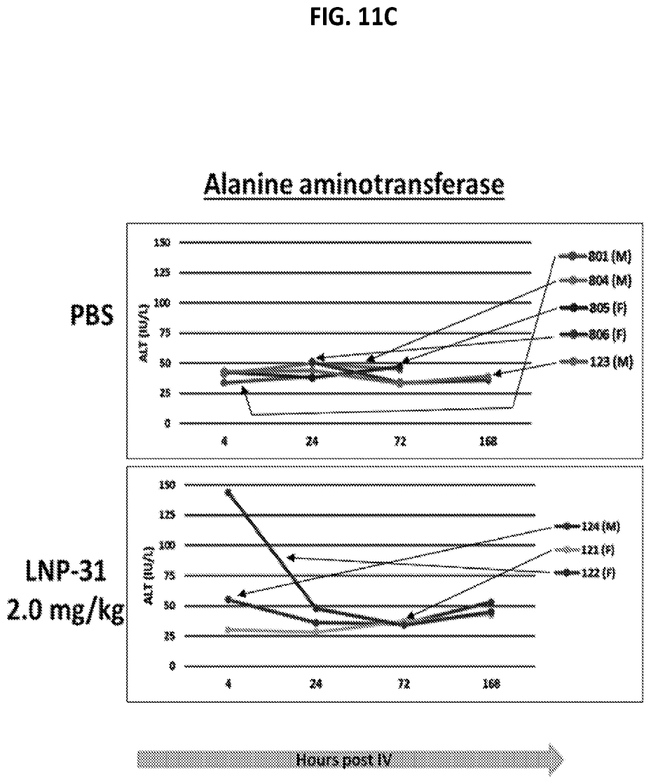

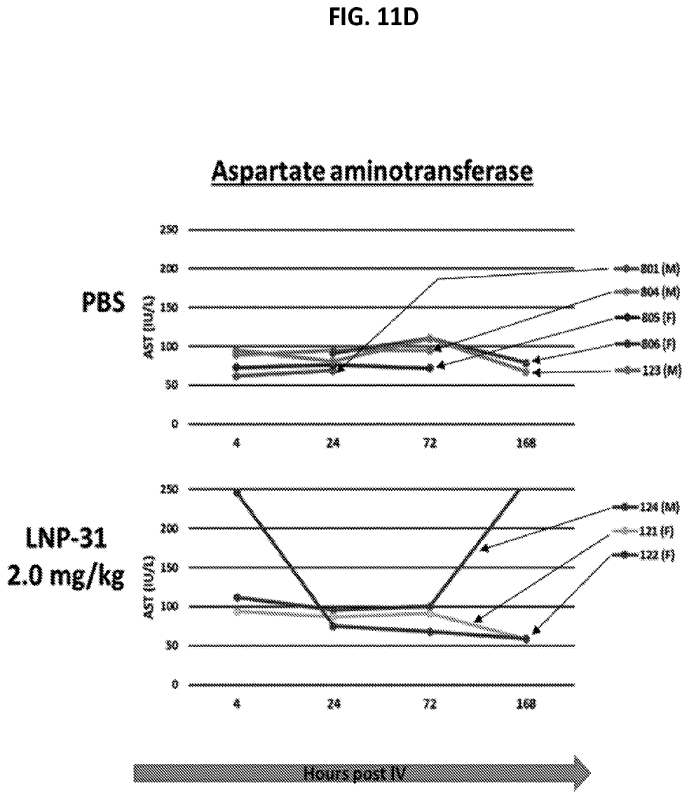

Next, we evaluated LNP toxicity in female Sprague Dawley rats using small and large lot (batch) LNP preparations. FIG. 11A shows a schematic overview of a study design for evaluating LNP toxicity in Sprague Dawley rats (n=4; 6-8 week-old female). On D −1 (i.e., 1 day prior to IV injection), body weight and baseline serum samples (blood) were collected for reference. On D 0, mice were injected intravenously (I.V.; via lateral tail vein injection) with LNP comprising B3 lipid, Cas9 mRNA/TTR sgRNA (4:1) at doses of 1.0, 2.0, 5.0 mg RNA per kg body weight. At 4- and 24-hours post I.V. injection body weight and blood samples (serum) were further collected for analysis. On D 7 (i.e., 7-days post IV injection) body weight measurements were taken, mice were necropsied, serum samples were collected, and sample tissues were collected. The readouts for thus study are viability and weight changes blood chemistry analysis (ALT, AST, ALK Phos, Bilirubin, blood urea nitrogen (BUN), and creatinine), cytokines analysis (IFN gamma, TNF alpha, IL-6, IL-1beta, IL-10, IL-18 CXCL1/2, MCP-1), liver injury biomarkers analysis (CD73, ARG1, GOT1, GSTa, SDH); and histopathology.

Systemic administration of LNP-B3 in Sprague-Dawley rats is well tolerated. Rats maintained consistent body weight, and no long term changes in liver enzymes FIG. 11B-D. All other serum chemistries were within normal limits. No remarkable findings were made upon histopathology examination of liver and spleen at Day 7 post IV.

Next, we evaluate B3-LNP in non-human primates (NHPs). First, we examine particle concentration (i.e., RNA concentration) in LNP batches, which are 0.6-0.9 mg/mL for preparation for NHP studies. Assuming a 5 kg rhesus macaque and a 0.7 mg/mL LNP prep (i.e., batch), a 2.0 mg/kg dose requires 14.3 mL of LNP and 3.0 mg/kg dose requires 21.4 mL of LNP. Additionally, we examine the freeze/thaw ability of the LNP formulation prior to evaluation in NHPs. Next, we examine endotoxin levels. The acceptable ranges for large animal dosing is less than 5 EU/kg for systemic administration. Assuming a 5 kg monkey, at a dose of 3.0 mg/kg and test article of 0.7 mg/mL, the maximum endotoxin level is 1.17 EU/mL. Assuming a 0.3 kg rat, dose is 5.0 mg/kg, for test article is 0.7 mg/mL the maximum endotoxin level is 0.70 EU/mL.

### Example 3. PCSK9-hE7 Mouse Line: Humanized Pcsk9 Gene for Pre-Clinical Gene Editing Studies

Here, we describe a further genome editing approach. The goal of genome editing is for the therapeutic effect to be durable and achieved in all patients independent of their mutation of the gene. For this purpose, we use the PCSK9 gene as a safe-harbor site and a meganuclease, ARCUS or Cas9, to target it.

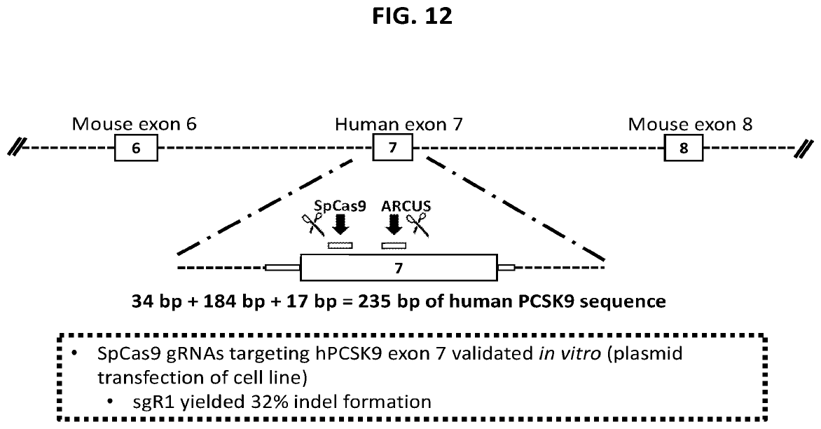

In this study, we perform pre-clinical gene editing studies using a PCSK9-hE7 mouse line having a humanized Pcsk9 gene. FIG. 12 shows a schematic overview of a study design in a PCSK9-hE7 mouse line having a humanized Pcsk9 gene using Arcus2 or Cas9 mRNA. This allows for studies to use renucleic acids which can be directly translated to NHP or human. PCSK9 gene is a “safe harbor” for insertion of a gene therapy mini-gene. Previously, the SpCas9 gRNAs targeting hPCSK9 exon 7 was validated in vitro (plasmid transfection of cell line) with sgR1 yielded 32% indel formation (data not shown).

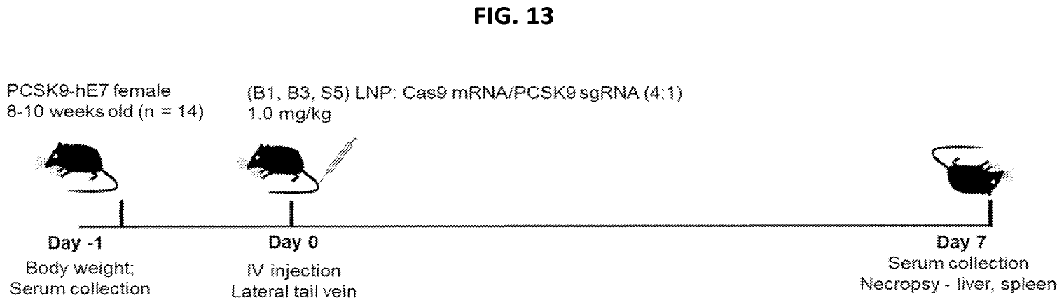

First, we perform study to evaluate hPCSK9 sgRNA (sgR1) in PCSK9-hE7 mice (female; 8-10 weeks old; n=14). FIG. 13 shows a schematic overview of a study design for evaluation of hPCSK9 sgRNA in PCSK9-hE7 mice. On Day-1, body weight measurement and serum samples are collected. On Day 0, mice are injected intravenously (I.V.; via lateral tail vein injection) with LNP comprising lipid (B1, B3, or S5), Cas9 mRNA/PCSK9 sgRNA (4:1) at dose of 1.0 mg RNA per kg body weight. On Day 7, serum samples are collected, mice are necropsied and sample tissues are collected (liver, spleen). Study groups are described in the Table 3, immediately below.

TABLE 3

| | RNA | Size | | Encapsulation | Endotoxin |
|---|---|---|---|---|---|
| LNP | (mg/mL) | (nm) | PDI | efficiency (%) | (EU/mL) |
| B1: SpCas9 | 0.64 | 132 | 0.03 | 39.5 | 16.5 |
| mRNA, PCSK9 | | | | | |
| sgRNA (4:1) | | | | | |
| B3: SpCas9 | 0.76 | 96 | 0.04 | 56.7 | 6.68 |
| mRNA, PCSK9 | | | | | |
| sgRNA (4:1) | | | | | |
| S5: SpCas9 | 0.88 | 96 | 0.09 | 68.1 | 6.29 |
| mRNA, PCSK9 | | | | | |
| sgRNA (4:1) | | | | | |

The readout in this study includes endotoxin level in LNP prep, levels of PCSK9 reduction evaluated by ELISA, percent indels evaluated by amplicon-seq, and overall complete In-life analysis. An exemplary sgRNA is sgRNA-sgr1 (Agilent 3′PACE): g*a*c*CAACUUUGGCCGCUGUGGUUUUAGAgcuagaaauagcAAGUUAAAAUAAGGCUA GUCCGUUAUCAacuugaaaaaguggcaccgagucggugcu #u #u #u (N=ribonucleotide; *=phosphorothioate bond; n=2′-O-methyl ribonucleotide; #=phosphonoacetate bond (PACE); SEQ ID NO: 1).

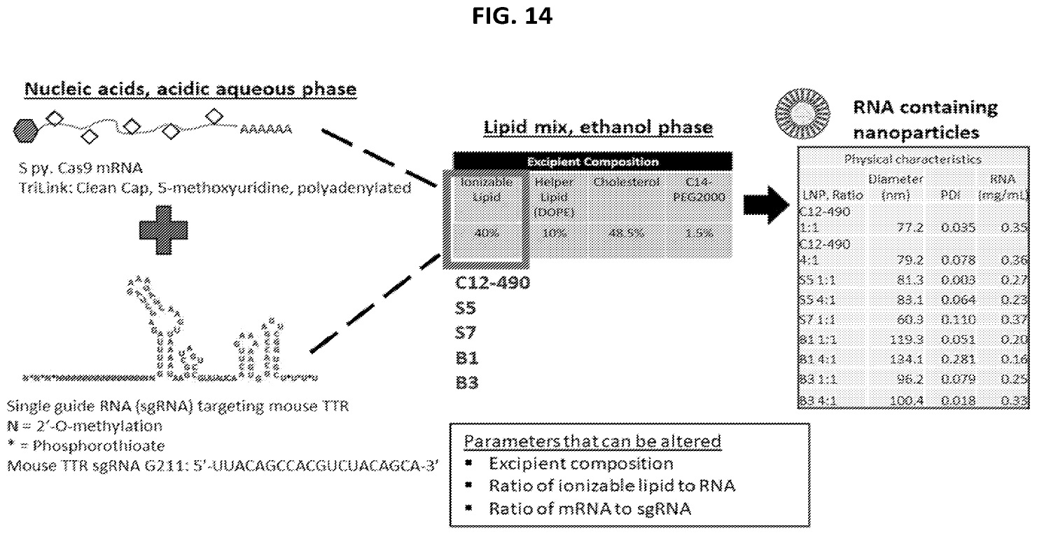

FIG. 14 shows a schematic overview for lipid nanoparticle (LNP) encapsulation of Cas9 mRNA and gene targeting sgRNA. An exemplary sgRNA is single guide RNA (sgRNA) targeting mouse TTR: Mouse TTR sgRNA G211: 5′-UUACAGCCACGUCUACAGCA-3 (N=2′-O-methylation; *=Phosphorothioate; SEQ ID NO: 2).

### Example 4. Purification Study

Blaze and TFF produced LNPs that are comparable to previously formulated LNPs. LNPs encapsulating Cas9 mRNA and mTTR sgRNA were formulated using the NanoAssemblr® Blaze™ (Precision Nanosystems) and either:
1) dialyzed against 1×PBS in dialysis cassettes and concentrated using Amicon centrifugal filters; or
2) processed using tangential flow filtration (TFF) to exchange buffer solution and concentrate LNPs.

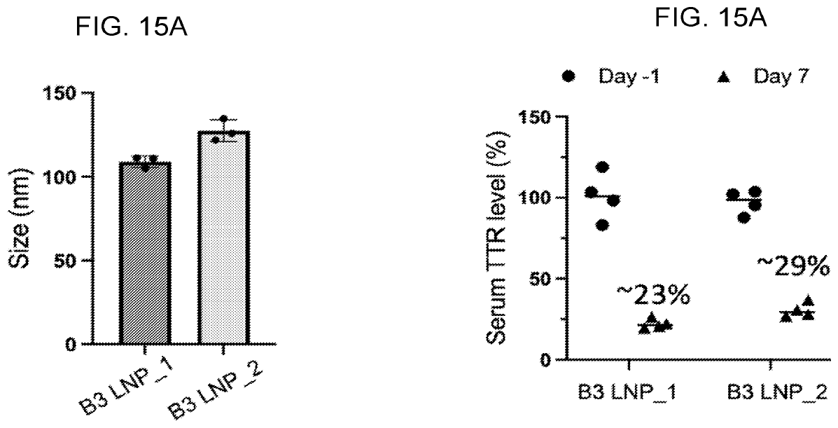

The resulting LNPs were characterized and injected to mice. Serum TTR levels in mice were measured at days −1 and 7. FIG. 15A shows that LNP size was comparable between the two methods. FIG. 15B shows that serum TTR levels were also consistent using either method.

### Example 5. Stabilization Study

Various batches of B3 LNPs were prepared. B3 LNP concentrated from normal centrifuge was used as the control. The following batches were prepared:
B3 (1): fresh LNP concentrated by TFF
B3 (2): control LNP concentrated by normal centrifuge
B3 (3): LNP concentrated by TFF then processed with cryoprotectant (sucrose)
B3 (4): LNP stored at fridge for 5 days
B3 (5): LNP stored at fridge for 7 days
B3 (6): frozen LNP after processing with sucrose for 7 days
LNP size was determined.

TABLE 4

| LNP | Size/nm | PDI | EE/% |
|---|---|---|---|
| B3_(1) | 143.3 ± 3.8 | 0.04 ± 0.03 | 64.7 |
| B3_(2) | 101.7 ± 0.7 | 0.15 ± 0.02 | 82.8 |

Endotoxin level was determined. Endotoxin level is acceptable for B3(1) batch, but the endotoxin level and encapsulation efficiency (EE) after adding sucrose were not acceptable (for B3(2)).

TABLE 5

| | | | Endotoxin | |
|---|---|---|---|---|
| Sample Name | Dilution | Score | concentration | |
| Standard #167 | 1:16,600 | + | 0.06 EU/mL = 2λ | |
| | 1:33,200 | + | 0.03 EU/mL = 1λ | |
| | 1:66,400 | + | 0.015 EU/mL = ½λ | |
| | 1:132,800 | − | 0.008 EU/mL = ¼λ | |
| | 0 | − | 0.00 | EU/mL |
| B3(1) | 1:5 | + | 0.15-0.99 | EU/mL |
| | 1:33 | − | | |
| | 1:50 | − | | |
| B3(2) | 1:5 | + | 0.99-1.5 | EU/mL |
| | 1:33 | + | | |
| | 1:50 | − | | |
| B3(3) | 1:5 | + | ≥1.5 | EU/mL |
| | 1:33 | + | | |
| | 1:50 | + | | |

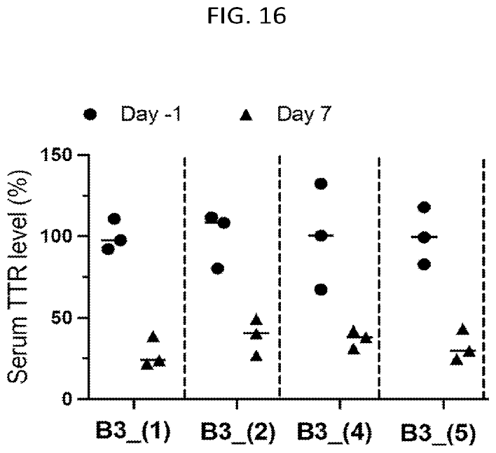

These LNP (other than B3(3)) were injected at 1mpk (total RNA) per dose at a concentration of 200 ng/μL. FIG. 16 shows that mTTR efficacy was similar even after storing the LNP in fridge for 7 days.

An endotoxin test was done for the following samples:
X1: LNP stored at fridge for 20 days
X2: LNP stored at fridge for 20 days then processed with sucrose (300 nM)
X3: sucrose solution (300 nM)

TABLE 6

| | | | Endotoxin | |
|---|---|---|---|---|
| Sample Name | Dilution | Score | concentration | |
| Standard #167 | 1:16,600 | + | 0.06 EU/mL = 2λ | |
| | 1:33,200 | + | 0.03 EU/mL = 1λ | |
| | 1:66,400 | + | 0.015 EU/mL = ½λ | |
| | 1:132,800 | − | 0.008 EU/mL = ¼λ | |
| | 0 | − | 0.00 | EU/mL |
| X1 | 1:5 | + | 0.15-0.99 | EU/mL |
| | 1:33 | − | | |
| | 1:50 | − | | |
| X2 | 1:5 | + | 0.15-0.99 | EU/mL |
| | 1:33 | − | | |
| | 1:50 | − | | |
| X3 | 1:5 | − | ≤1.5 | EU/mL |
| | 1:33 | − | | |
| | 1:50 | − | | |

Acceptable endotoxin level was found for all samples.

### Example 6. Encapsulation Study

B3 LNP loading Cas9/mTTR was formulated by Blaze/TFF, to assess encapsulation efficiency. The following samples were tested.
B3 LNP concentrated from normal centrifuge as control
B3_(1): fresh LNP concentrated by TFF
B3_(2): control LNP concentrated by normal centrifuge

TABLE 7

| LNP | Size/nm | PDI | EE/% |
|---|---|---|---|
| B3_(1) | 129.4 ± 1.8 | 0.07 ± 0.03 | 70.1 |
| B3_(2) | 114.1 ± 1.4 | 0.06 ± 0.01 | 83.1 |

It was determined that the low EE might come from the degradation of mRNA as TFF process take 6 h at room temperature. Thus, a Larger cartridge column was employed, to shorten the buffer exchange time.

TABLE 8

| LNP | Size/nm | PDI | EE/% |
|---|---|---|---|
| B3_(1) | 106.9 ± 2.4 | 0.16 ± 0.01 | 78.6 |
| B3_(2) | 104.3 ± 3.2 | 0.18 ± 0.03 | 84.3 |

After shortening buffer exchange time at a relative low temperature for TFF, improvement in encapsulation efficacy was seen.

### Example 7. Evaluation of B3 LNP for Cas9-TTR Editing in Mice

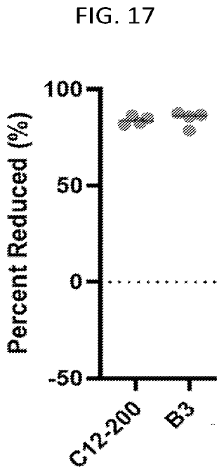

In this study, C57BL/6J mice (male at 6-8 weeks old; n=4/group) were used. On D −1 (i.e., 1 day prior to IV injection), body weight and baseline serum samples were collected for reference. On D 0, mice were injected intravenously (I.V.; via lateral tail vein injection) with LNP comprising C12-200 or B3 lipid, Cas9 mRNA/TTR sgRNA (4:1) at a dose of 1.0 mg RNA per kg body weight. On D 7 (i.e., 7-days post IV injection) body weight measurements were taken, mice were necropsied, serum samples were collected, and sample tissues were collected (spleen, heart, lung, kidney and muscle). Collected samples were analyzed for gene-editing kinetics by ELISA (i.e., serum TTR levels). FIG. 17 demonstrates gene editing efficiency of B3 is similar to the C12-200 benchmark.

The disclosures of each and every patent, patent application, and publication cited herein are hereby incorporated herein by reference in their entirety. U.S. Provisional Patent Application Nos. 63/364,859, 63/330,972, and 63/331,060 are incorporated herein in their entireties. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising biodegradable lipid nanoparticles (LNP) useful for delivering a nucleic acid to a liver cell, the LNP being formed from:
(a) at least one ionizable lipid compound having the structure of Formula (LA), Formula (IB), or combinations thereof: 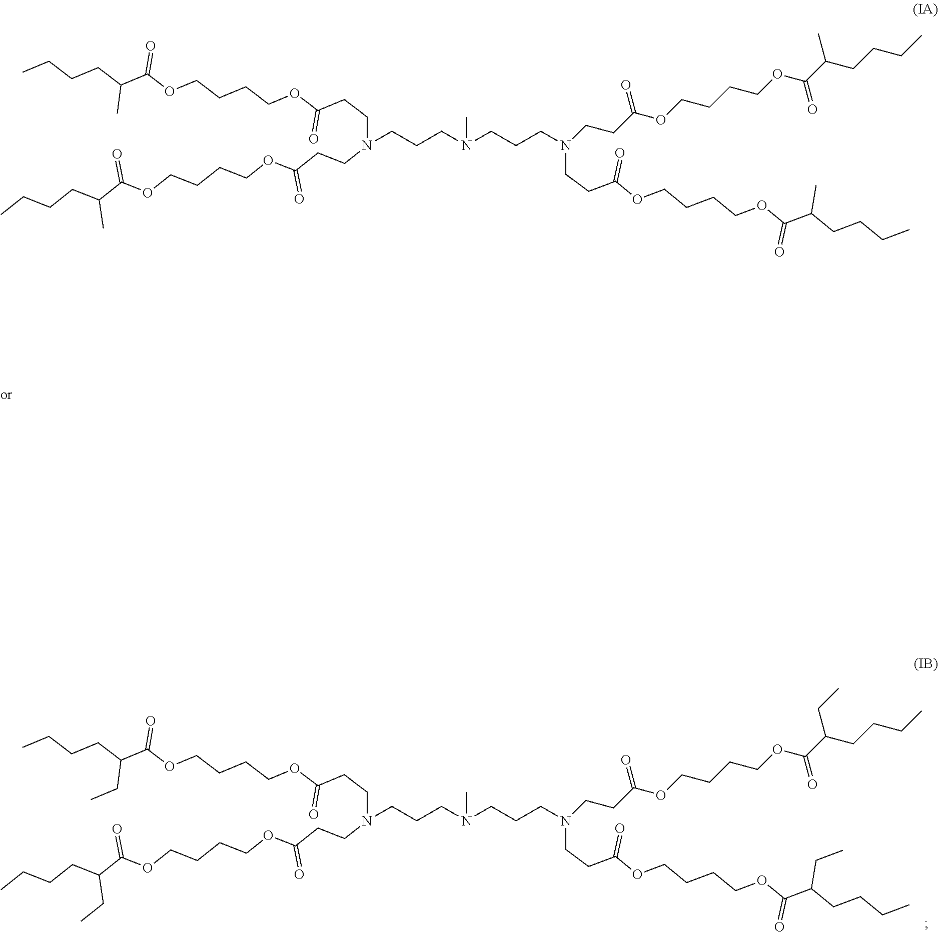
or a racemate, an enantiomer, a diastereomer, a pharmaceutically acceptable salt, or a derivative thereof;
wherein the total concentration of ionizable lipid(s) (a) in the LNP is present in a concentration range of about 1 mol % to about 99 mol %, based on the total amount of lipids in the LNP;
(b) at least one neutral phospholipid, wherein the neutral phospholipid is present in a concentration range of about 10 mol % to about 45 mol % based on the total amount of lipids in the LNP;
(c) at least one cholesterol lipid, wherein the total cholesterol lipid is in a concentration range of about 5 mol % to about 55 mol % based on the total amount of lipids in the LNP; and
(d) at least one polyethylene glycol (PEG) lipid, wherein the total PEG-lipid is in a concentration range of about 0.5 mol % to about 12.5 mol % based on the total amount of lipids in the LNP;
(e) at least one nucleic acid encapsulated in the LNP.

2. The composition of claim 1, wherein the ionizable lipid has the structure of (IB), or a derivative thereof

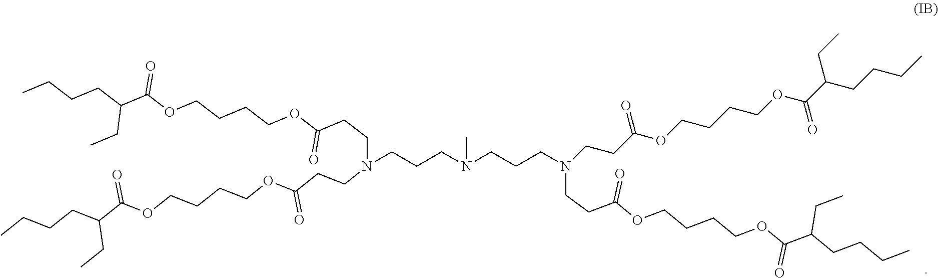 [SMILES: CCCCC(CC)C(=O)OCCCCOC(=O)CCN(CCCN(C)CCCN(CCC(=O)OCCCCOC(=O)C(CC)CCCC)CCC(=O)OCCCCOC(=O)C(CC)CCCC)CCC(=O)OCCCCOC(=O)C(CC)CCCC]

3. The composition of claim 1, wherein the ionizable lipid has the structure of (IA), or a derivative thereof

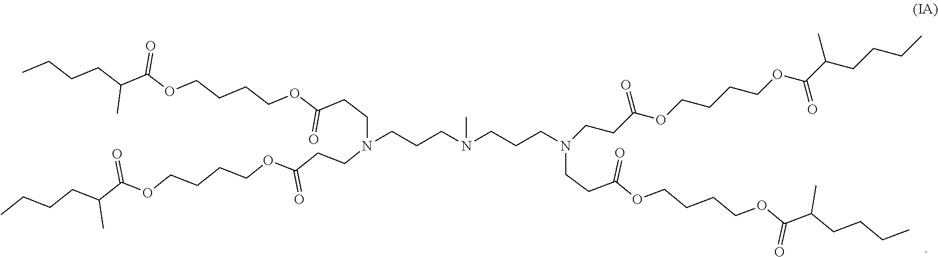 [SMILES: CCCCC(C)C(=O)OCCCCOC(=O)CCN(CCCN(C)CCCN(CCC(=O)OCCCCOC(=O)C(C)CCCC)CCC(=O)OCCCCOC(=O)C(C)CCCC)CCC(=O)OCCCCOC(=O)C(C)CCCC]

4. The composition of claim 1, wherein the neutral phospholipid comprises dioleoylphosphatidylethanolamine (DOPE).

5. The composition of claim 1, wherein the cholesterol lipid is a cholesterol or a cholesteryl derivate.

6. The composition of claim 1, wherein the PEG-lipid comprises C12-PEG2000 or C12-PEG490.

7. The composition of claim 1, wherein the ratio of ionizable lipid to nucleic acid in an LNP is about 5:1 to about 10:1, based on weight percentage of the lipids.

8. The composition of claim 1, wherein the molar (mol) ratio of a:b:c:d is about 35%:16%:46.5%:2.5%.

9. The composition of claim 1, wherein the nucleic acid is DNA or RNA.

10. The composition according to claim 1, wherein the average diameter of the LNPs comprises about 50 nm to about 150 nm as determined using cryo-transmission electron microscopy (TEM).

11. The composition according to claim 1, wherein the nucleic acid comprises a coding sequence for an editing enzyme.

12. The composition according to claim 11, wherein the nucleic acid is an mRNA encoding a Cas9.

13. (canceled)

14. The composition according to claim 11, wherein the LNP further comprises an sgRNA.

15. The composition according to claim 14, wherein the ratio of mRNA to sgRNA is about 1:5 to 5:1 mRNA to sgRNA based on molar percentage.

16. (canceled)

17. The composition according to claim 11, wherein the nucleic acid encodes a synthetic or engineered nuclease, a zinc finger nuclease, a TAL-effector nuclease, or a meganuclease.

18. The composition according to claim 17, wherein the nuclease targets transthyretin (TTR), albumin, or PCSK9.

19. The composition according to claim 1, wherein the composition further comprises a second nucleic acid that encodes a therapeutic transgene.

20. The composition according to claim 19, wherein the therapeutic transgene is associated with a liver enzyme disorder, a lysosomal storage disorder, a glycogen storage disease or deficiency, a urea cycle disorder, or a lipid disorder.

21. The composition according to claim 19, wherein the second nucleic acid comprises a liver-specific promoter operably linked to a sequence encoding a therapeutic gene product or a transcript thereof.

22. The composition according to claim 1, further comprising about 300 nM sucrose.

23. A method of delivering a gene product to a subject in need thereof, the method comprising administering a therapeutically effectively amount of at least one biodegradable LNP composition of claim 1, wherein the nucleic acid encodes a gene product or a transcript therefor to the subject.

24. The method according to claim 23, wherein the method further comprises co-administering a gene therapy vector with the biodegradable LNP composition.

25-29. (canceled)
